(19) United States
(12) Patent Application Publication
(10) Pub. No.: US 2025/0043172 A1
(43) Pub. Date: Feb. 6, 2025

(54) BINUCLEAR PLATINUM COMPLEX LUMINESCENT MATERIAL AND APPLICATION THEREOF

(71) Applicant: GUANGDONG AGLAIA OPTOELECTRONIC MATERIALS CO., LTD, Foshan (CN)

(72) Inventors: Huiyang Li, Foshan (CN); Xinwei Wu, Foshan (CN); Lei Dai, Foshan (CN); Lifei Cai, Foshan (CN)

(21) Appl. No.: 18/715,205

(22) PCT Filed: Dec. 18, 2022
(86) PCT No.: PCT/CN2022/139820

§ 371 Date: May 31, 2024

(30) Foreign Application Priority Data
Dec. 22, 2021 (CN) 202111577311.0
Nov. 29, 2022 (CN) 202211510555.1

(51) Int. Cl. C09K 11/06; H10K 50/11; H10K 85/30

(52) U.S. Cl. CPC C09K 11/06; H10K 50/11; H10K 85/346; H10K 85/361; C09K 2211/185

(57) ABSTRACT

The present disclosure relates to a binuclear platinum complex luminescent material and application thereof. The binuclear platinum complex luminescent material has the structure of chemical formula (I), exhibiting relatively good luminous efficiency and device lifetime when applied to an organic light-emitting diode. The present disclosure also provides an organic electroluminescent device, including a cathode, an anode, and an organic layer. The organic layer is one or more of a hole injection layer, a hole transport layer, a light-emitting layer, a hole blocking layer, an electron transport layer, and an electron injection layer. At least one of the organic layers includes the compound of structural formula (I).

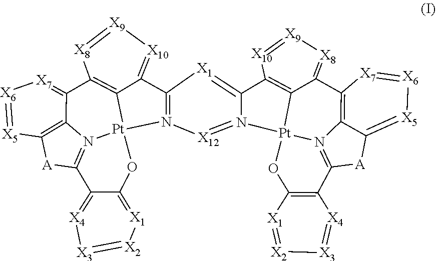

### CROSS-REFERENCE TO RELATED APPLICATIONS

This application is an U.S. national phase application under 35 U.S.C. § 371 based upon international patent application No. PCT/CN2022/139820, filed on Dec. 18, 2022, which itself claims priority to Chinese patent application No. 2021115773110, filed on Dec. 22, 2021 and Chinese patent application No. 2022115105551, filed on Nov. 29, 2022. The contents of the above-identified applications are hereby incorporated herein in their entireties by reference.

### TECHNICAL FIELD

The present disclosure relates to the field of luminescent materials, and particularly relates to a class of binuclear platinum complexes and their application in organic light-emitting diodes.

### BACKGROUND

Organic optoelectronic devices include but are not limited to the following categories: organic light-emitting diodes (OLEDs), organic thin film transistors (OTFTs), organic photovoltaic devices (OPVs), light-emitting electrochemical cells (LCEs) and chemical sensors.

In recent years, OLEDs, as a lighting and display technology with huge application prospects, have attracted widespread attention from academia and industry. OLED devices possess the characteristics of self-illumination, wide viewing angle, short response time and can be made into flexible devices, becoming strong competitors in the next generation display and lighting technologies. However, OLEDs currently still have problems such as low efficiency and short lifetime, which require further research.

Early fluorescent OLEDs typically can utilize only singlet state emission. Triplet excitons generated in the device cannot be effectively utilized and return to the ground state through non-radiative means, which limits the widespread application of OLEDs. In 1998, Zhi Zhiming et al. from the University of Hong Kong firstly reported the electro-phosphorescence phenomenon. In the same year, Thompson et al. used transition metal complexes as luminescent materials to prepare phosphorescent OLEDs. The phosphorescent OLEDs can efficiently utilize singlet and triplet excitons to emit light, and can theoretically achieve 100% internal quantum efficiency, greatly promoting the commercialization process of OLEDs. The emission colors of OLEDs can be controlled through structural design of the luminescent materials. OLEDs can include one emitting layer or multiple emitting layers to achieve a desired spectrum. Currently, green, yellow, and red phosphorescent materials have been commercialized. Commercial OLED displays usually use a combination of blue fluorescence and yellow or green and red phosphorescence to achieve full-color display, but there is a pressing need in the industry for luminescent materials with higher efficiency and longer service life. Metal complex luminescent materials have been widely used in the organic light-emitting display industry, but their performance, such as luminous efficiency and excited state lifetime, still need to be further improved. At present, compared with vapor-deposited luminescent materials, the development of high-performance metal complexes suitable for solution-processed devices is relatively lagging behind, which has become an important factor limiting the development of solution-processed devices.

### SUMMARY

In view of the above issues existing in the related art, the present disclosure provides a class of binuclear platinum complex luminescent materials. These materials, when used in organic light-emitting diodes, exhibit relatively good luminous efficiency and device lifetime.

The present disclose further provides an organic light-emitting diode including the binuclear platinum complex.

The binuclear platinum complex material is a compound having a structure of formula (I):

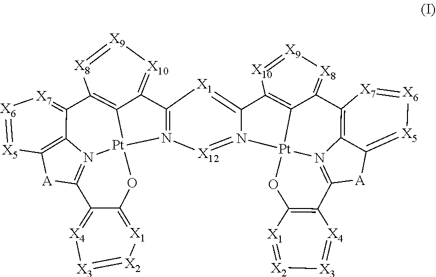

wherein:
X₁ to X₁₂ are each independently selected from N or CR;
A is selected from CR¹ R², NR³, O, S, or Se;
R, R¹, R², and R³ are each independently selected from the following groups: hydrogen, deuterium, halogen, amino group, C1-C20 alkylcarbonyl, carboxyl, aldehyde, C1-C20 thioalkyl, cyano group, sulfonyl, phosphino group, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted alkoxy having 1 to 20 carbon atoms, substituted or unsubstituted aryl having 6 to 30 carbon atoms, or substituted or unsubstituted heteroaryl having 3 to 30 carbon atoms; or any two adjacent R, R¹, R², and R³ are connected to form a ring;
the heteroatom in the heteroaryl is one or more of N, S, or O;
the substitution is by halogen, amino group, cyano group, C6-C12 aryl, or C1-C4 alkyl.

In an embodiment, R, R¹, R², and R³ are each independently selected from hydrogen, deuterium, halogen, amino group, C1-C6 thioalkyl, cyano group, substituted or unsubstituted alkyl having 1 to 6 carbon atoms, substituted or unsubstituted cycloalkyl having 3 to 6 ring carbon atoms, substituted or unsubstituted alkenyl having 2 to 6 carbon atoms, substituted or unsubstituted alkoxy having 1 to 6 carbon atoms, substituted or unsubstituted aryl having 6 to 12 carbon atoms, or substituted or unsubstituted heteroaryl having 3 to 6 carbon atoms.

In an embodiment, R, R¹, R², and R³ are each independently selected from hydrogen, deuterium, halogen, cyano group, C1-C4 alkyl, substituted or unsubstituted cycloalkyl having 3 to 6 ring carbon atoms, substituted or unsubstituted aryl having 6 to 12 carbon atoms, or substituted or unsubstituted heteroaryl having 3 to 6 carbon atoms;

the substitution is by phenyl or C1-C4 alkyl.

In an embodiment, A is selected from NR³, O, or S.

In an embodiment, R, R¹, R², and R³ are each independently selected from hydrogen, deuterium, cyano group, methyl, isopropyl, isobutyl, tert-butyl, substituted or unsubstituted cyclopentyl, substituted or unsubstituted cyclohexyl, substituted or unsubstituted phenyl, substituted or unsubstituted pyridyl, substituted or unsubstituted pyrazinyl, or substituted or unsubstituted pyrimidinyl.

In an embodiment, the formula (I) is the following structure:

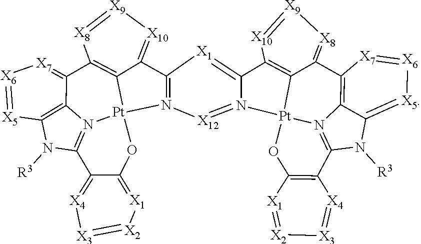

R and R³ are independently selected from hydrogen, deuterium, methyl, tert-butyl, substituted or unsubstituted cyclopentyl, substituted or unsubstituted cyclohexyl, substituted or unsubstituted phenyl, or substituted or unsubstituted pyridyl.

In an embodiment, the formula (I) is the following structure:

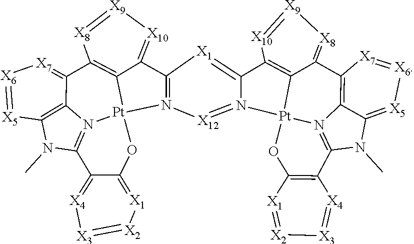

R is selected from hydrogen, deuterium, methyl, tert-butyl, substituted or unsubstituted cyclopentyl, substituted or unsubstituted cyclohexyl, substituted or unsubstituted phenyl, or substituted or unsubstituted pyridyl.

In an embodiment, at most one of X₁ to X₄ is N, and the rest are CR;

at most one of X₅ to X₇ is N, and the rest are CR;

at most one of X₈ to X₁₀ is N, and the rest are CR; X₁₂ is CH, and X₁₁ is N or CH.

In an embodiment, X₁, X₃, and X₉ are CR; X₂, X₄ to X₈, and X₁₀ are CH.

In an embodiment, R is hydrogen, deuterium, methyl, tert-butyl, cyclopentyl, cyclohexyl, phenyl, or pyridyl.

Examples of the platinum metal complex according to the present disclosure are listed below, but they are not limited to the listed structures:

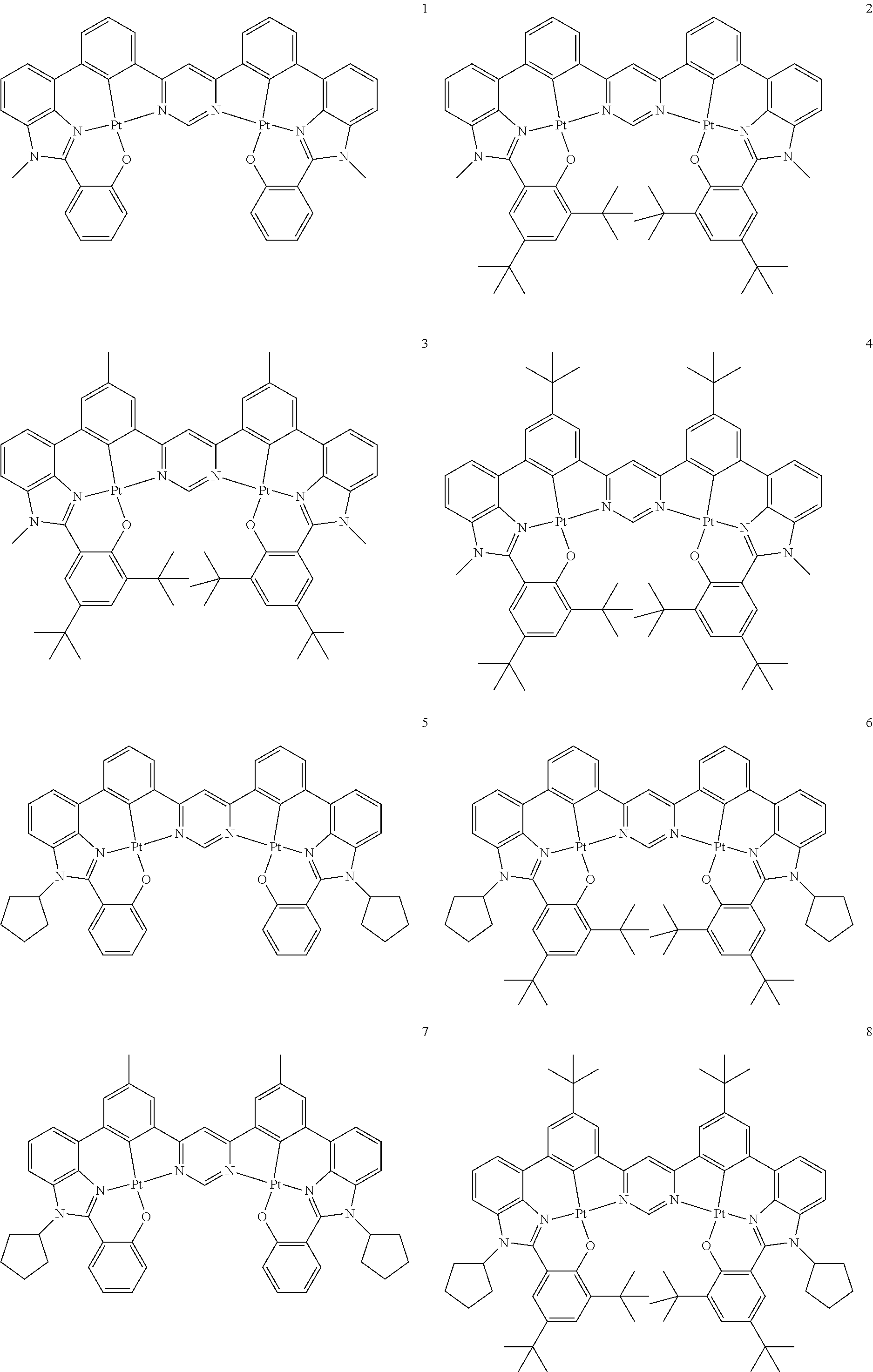

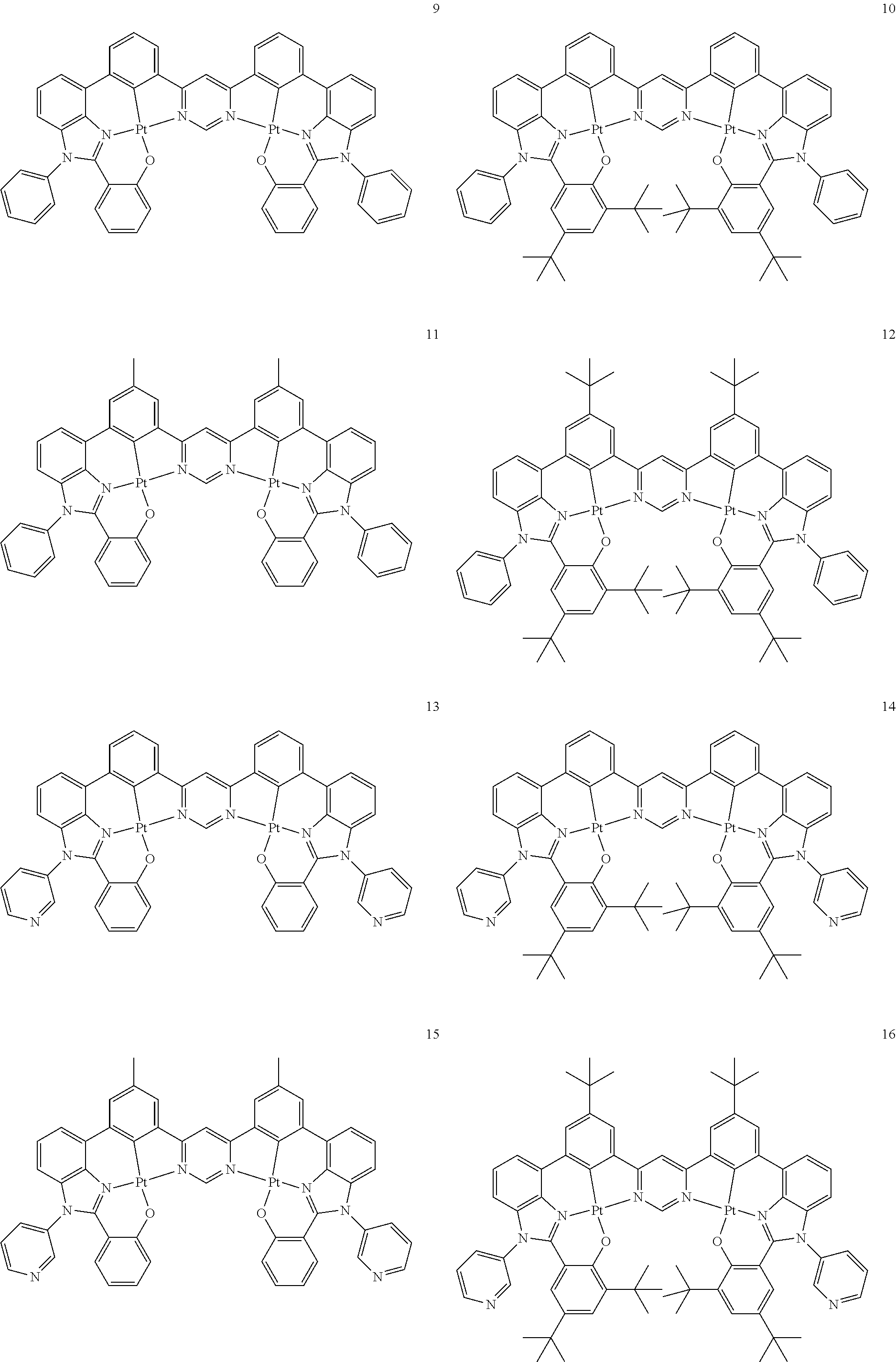

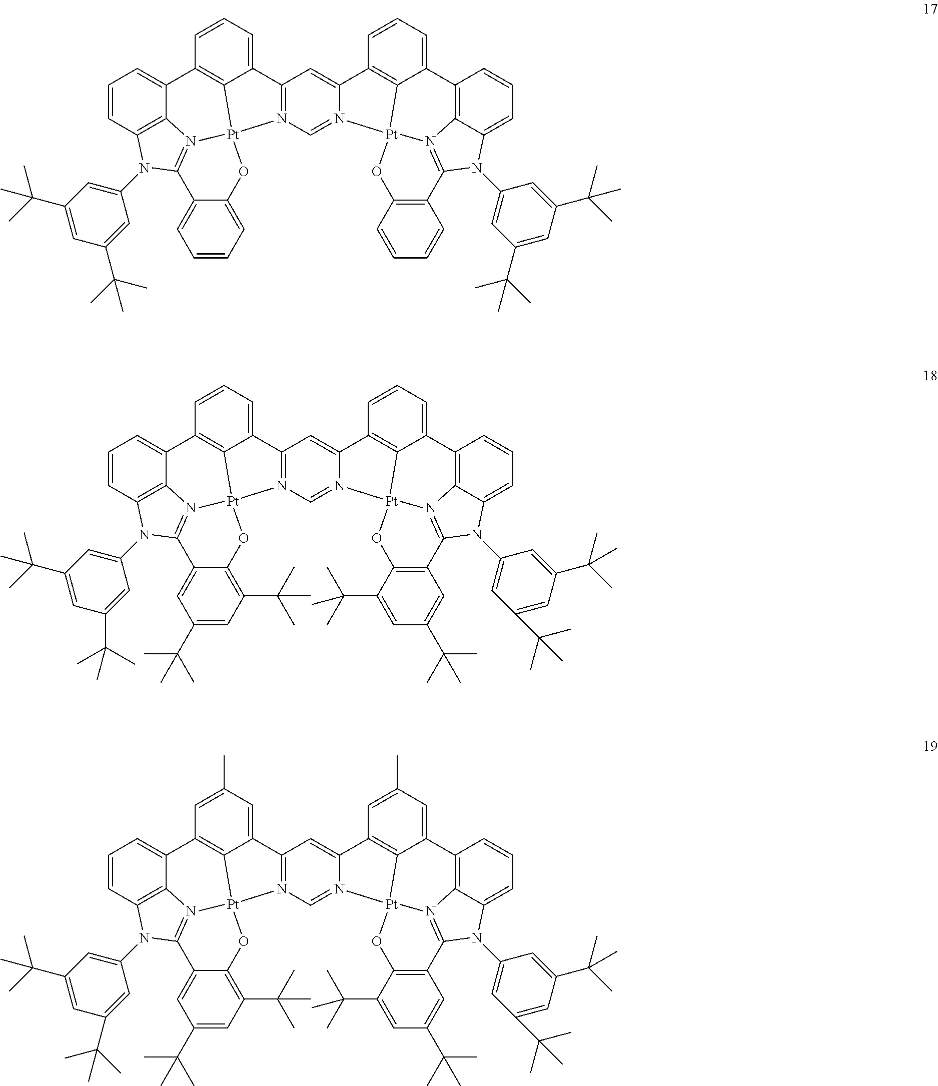

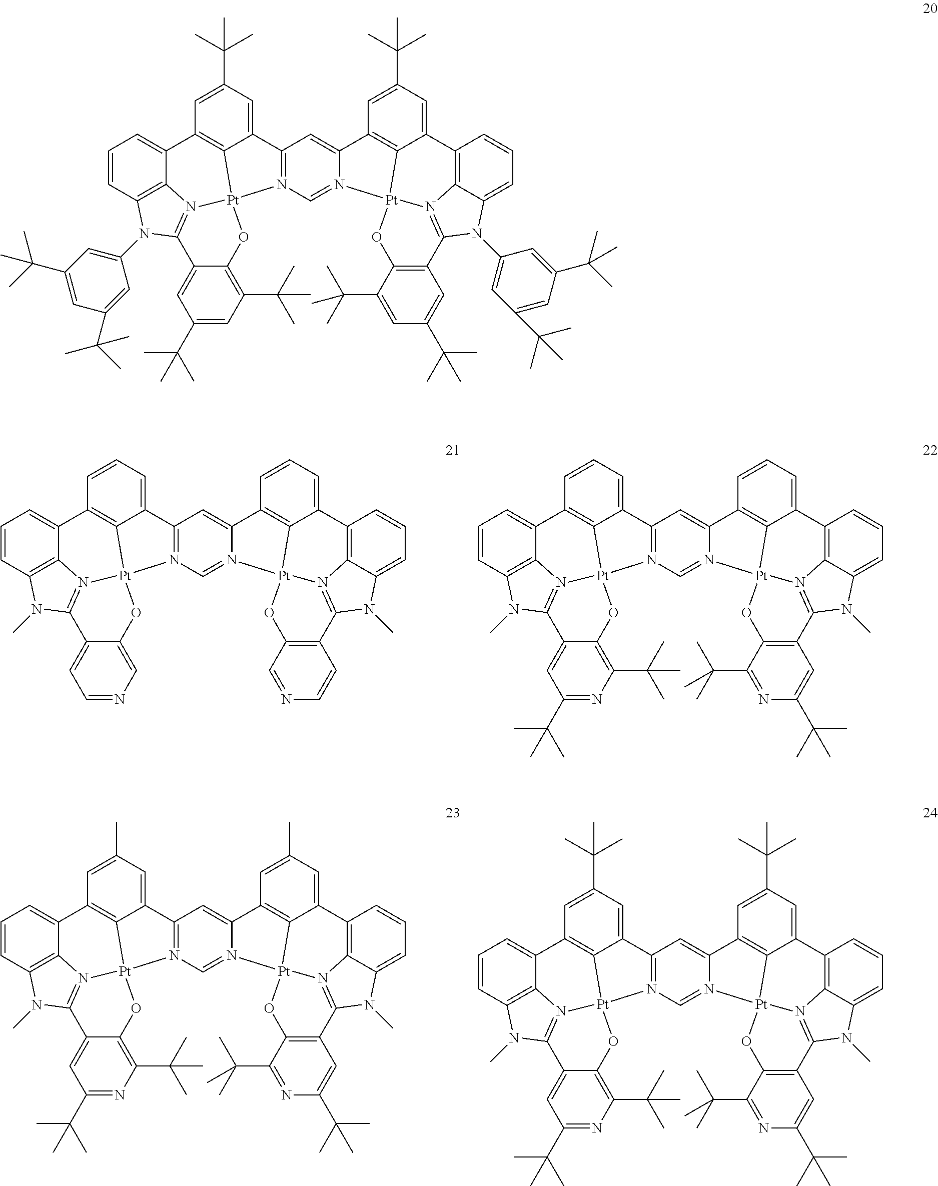

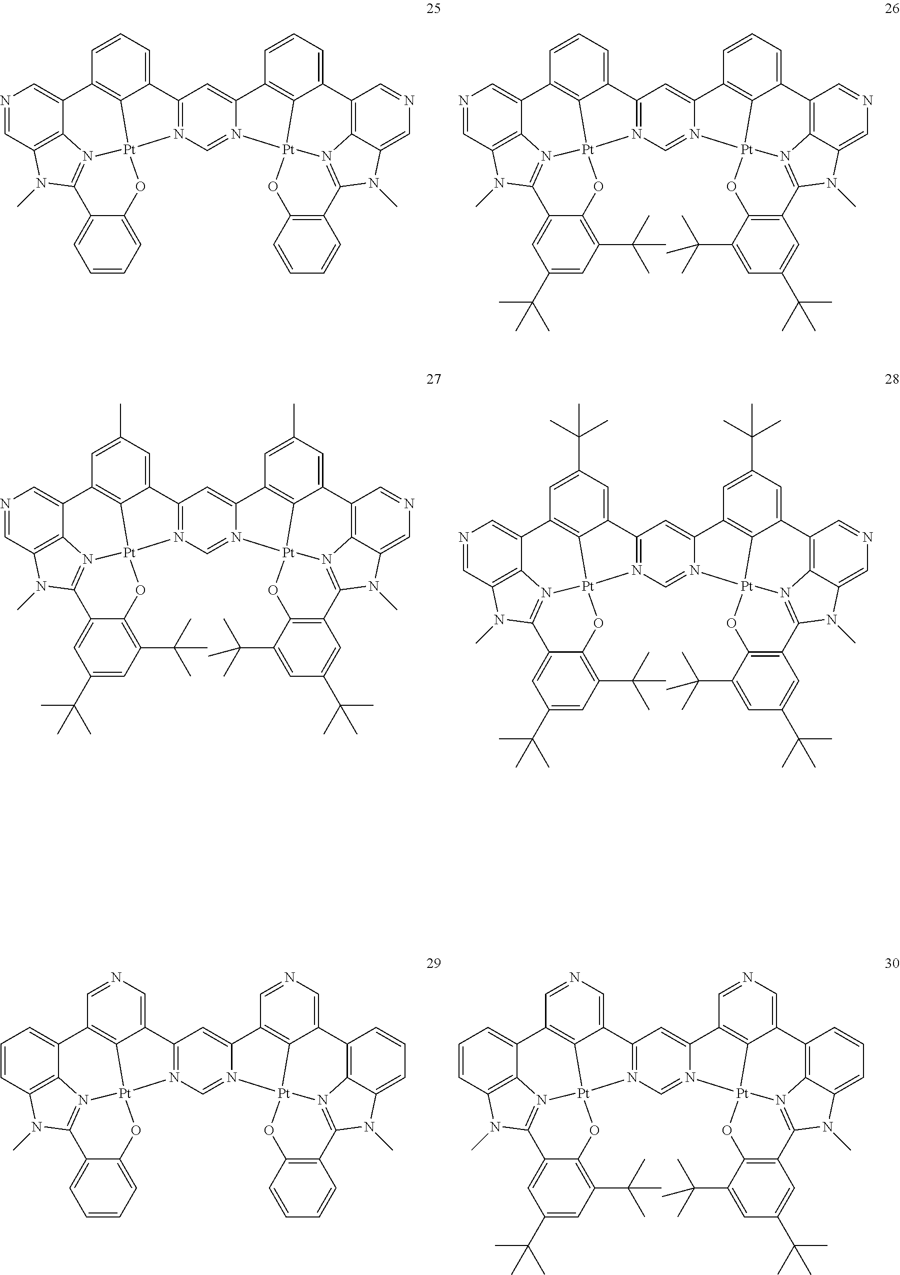

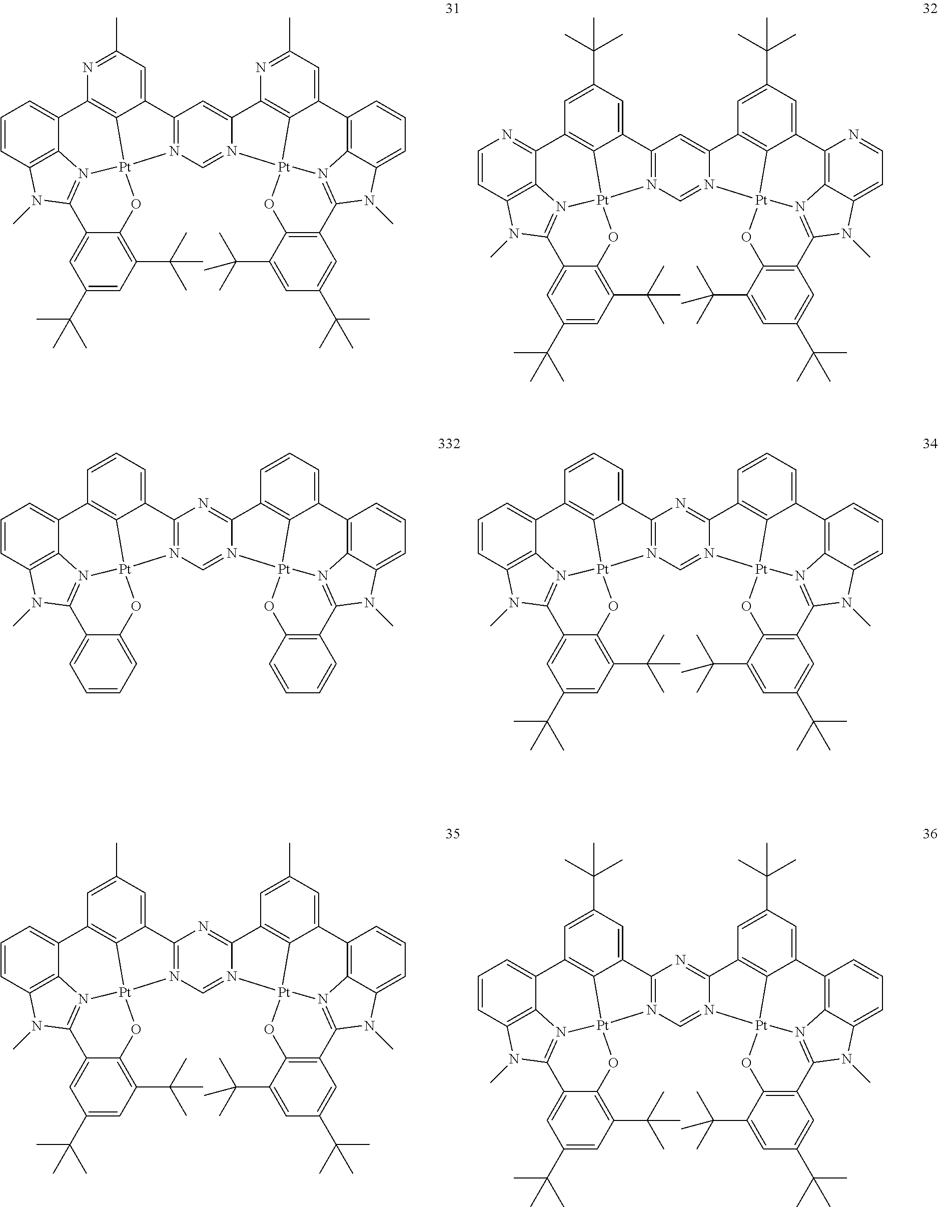

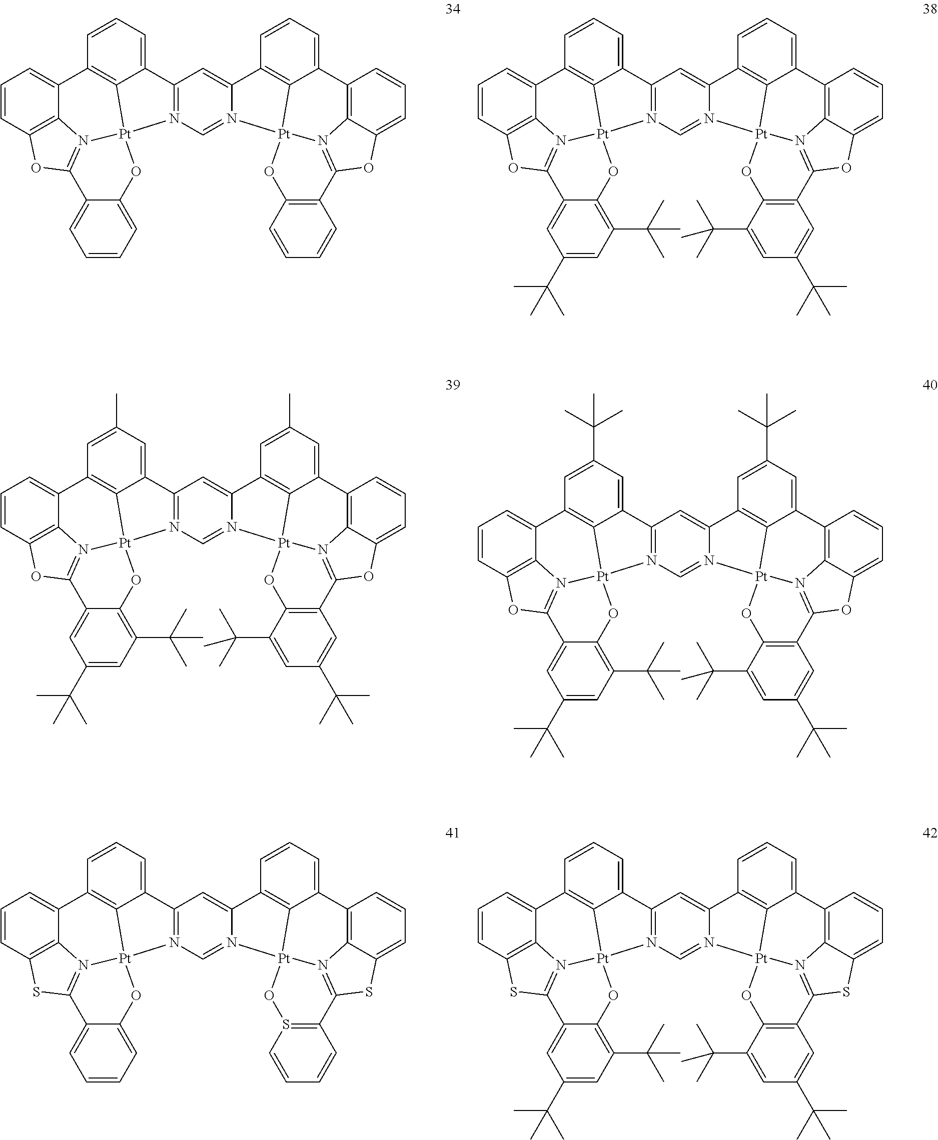

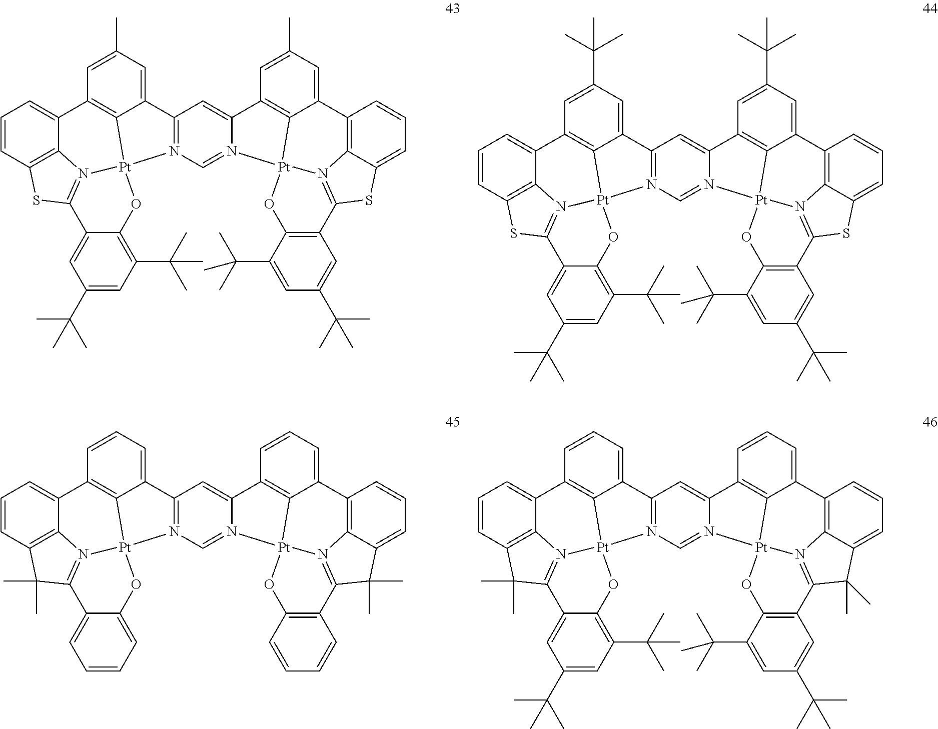

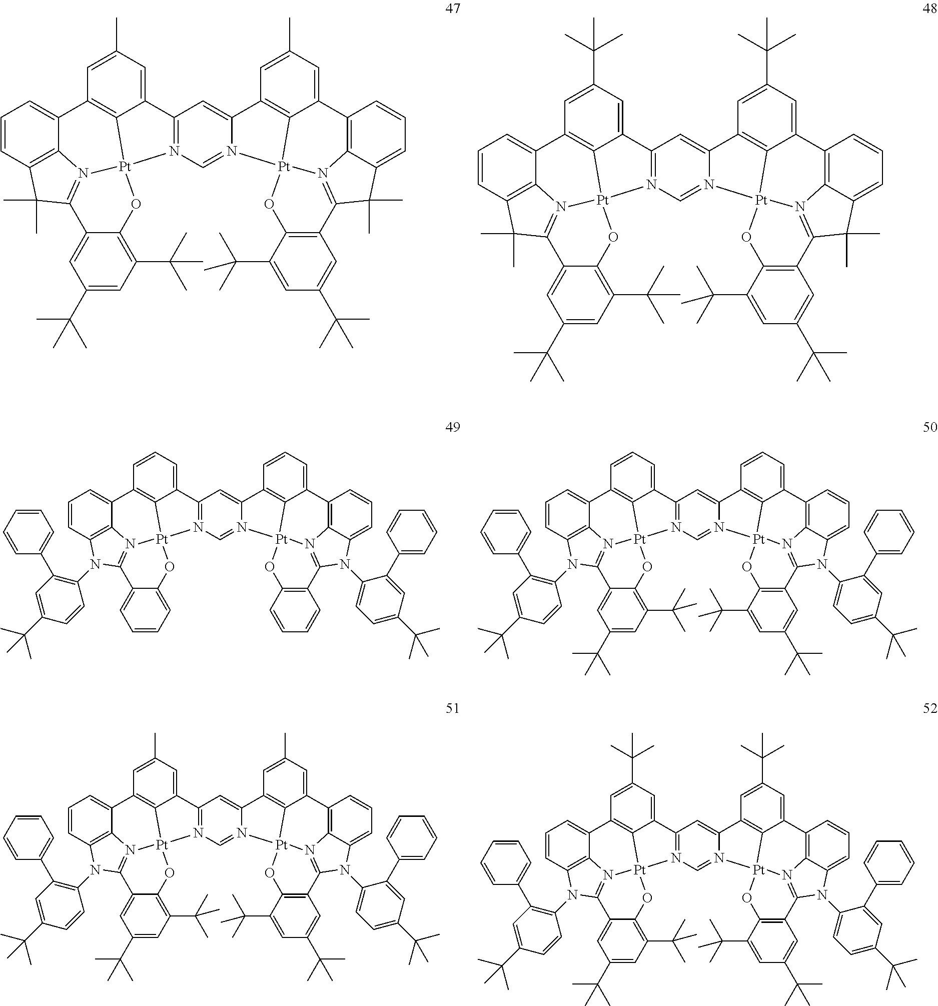

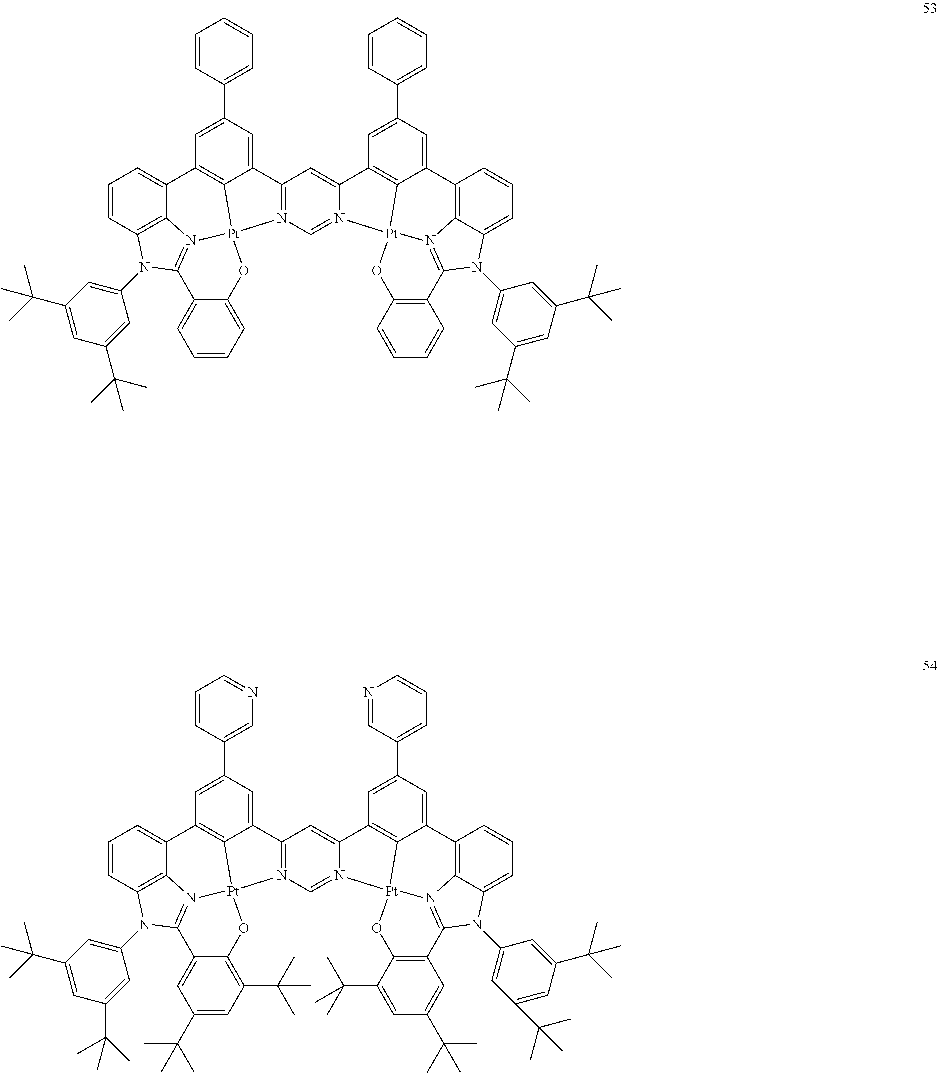

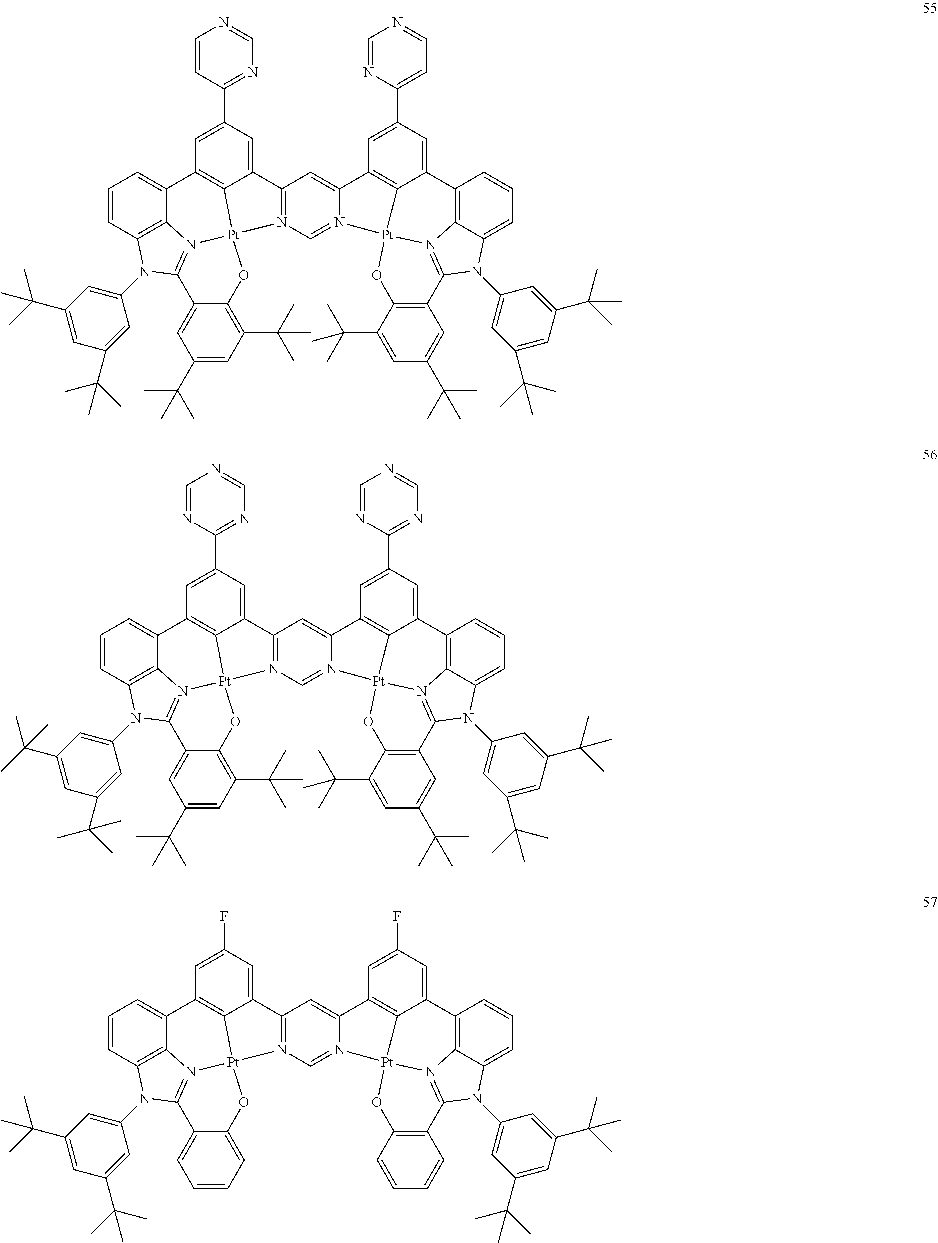

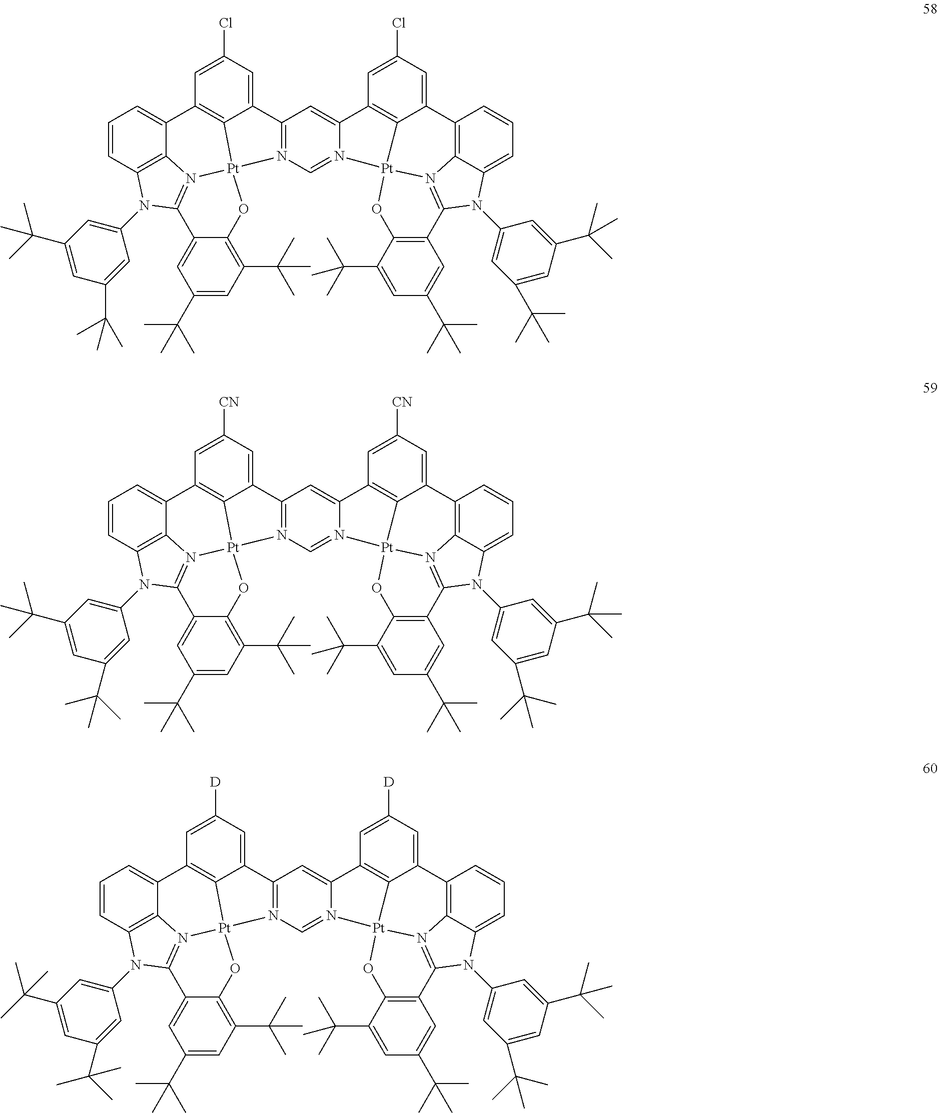

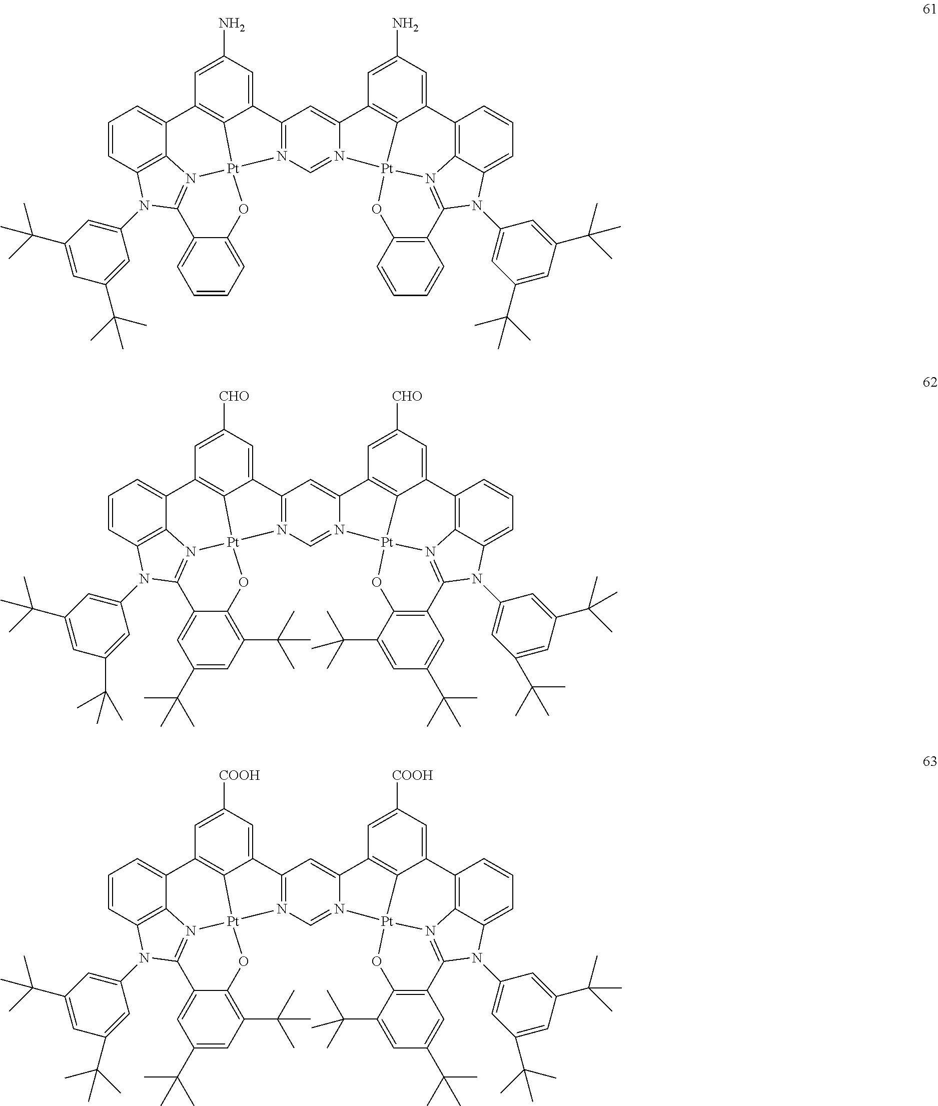

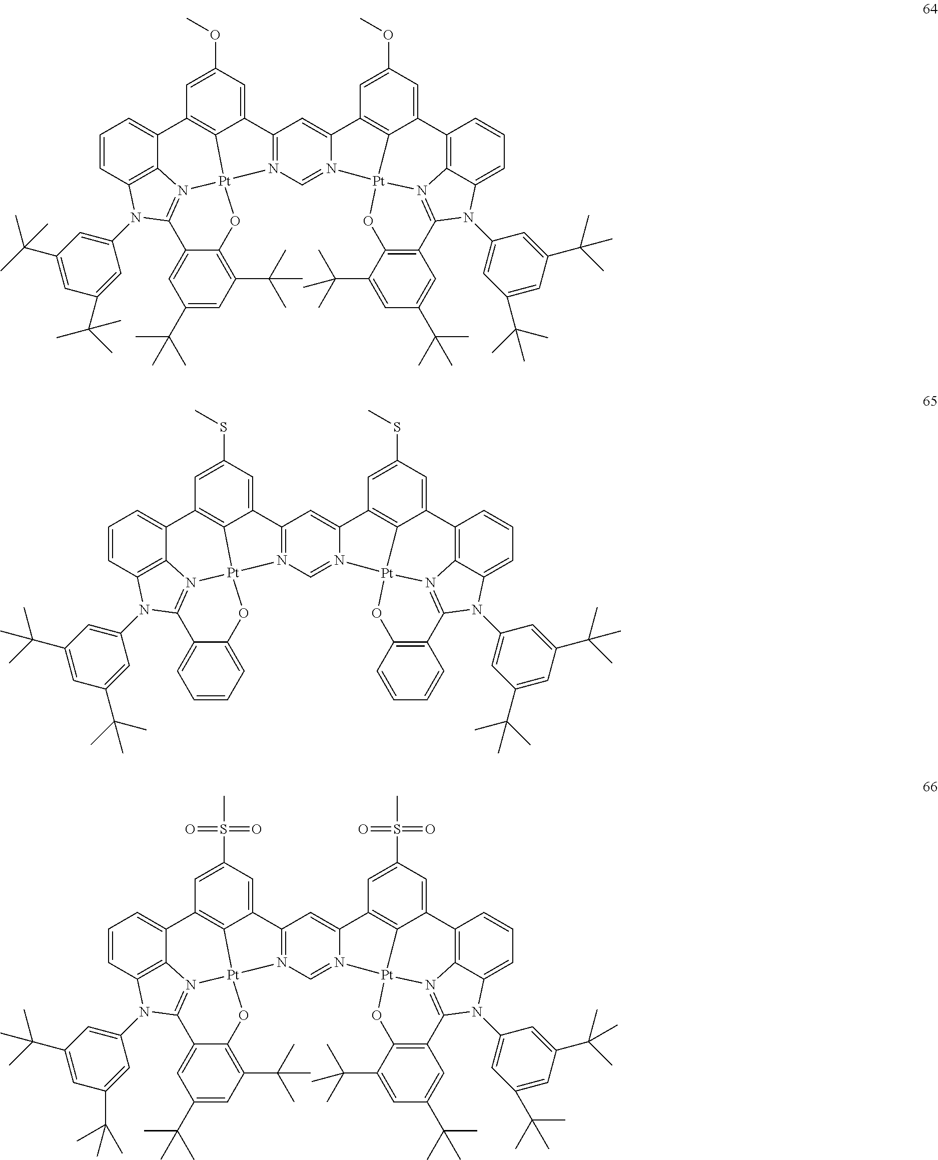

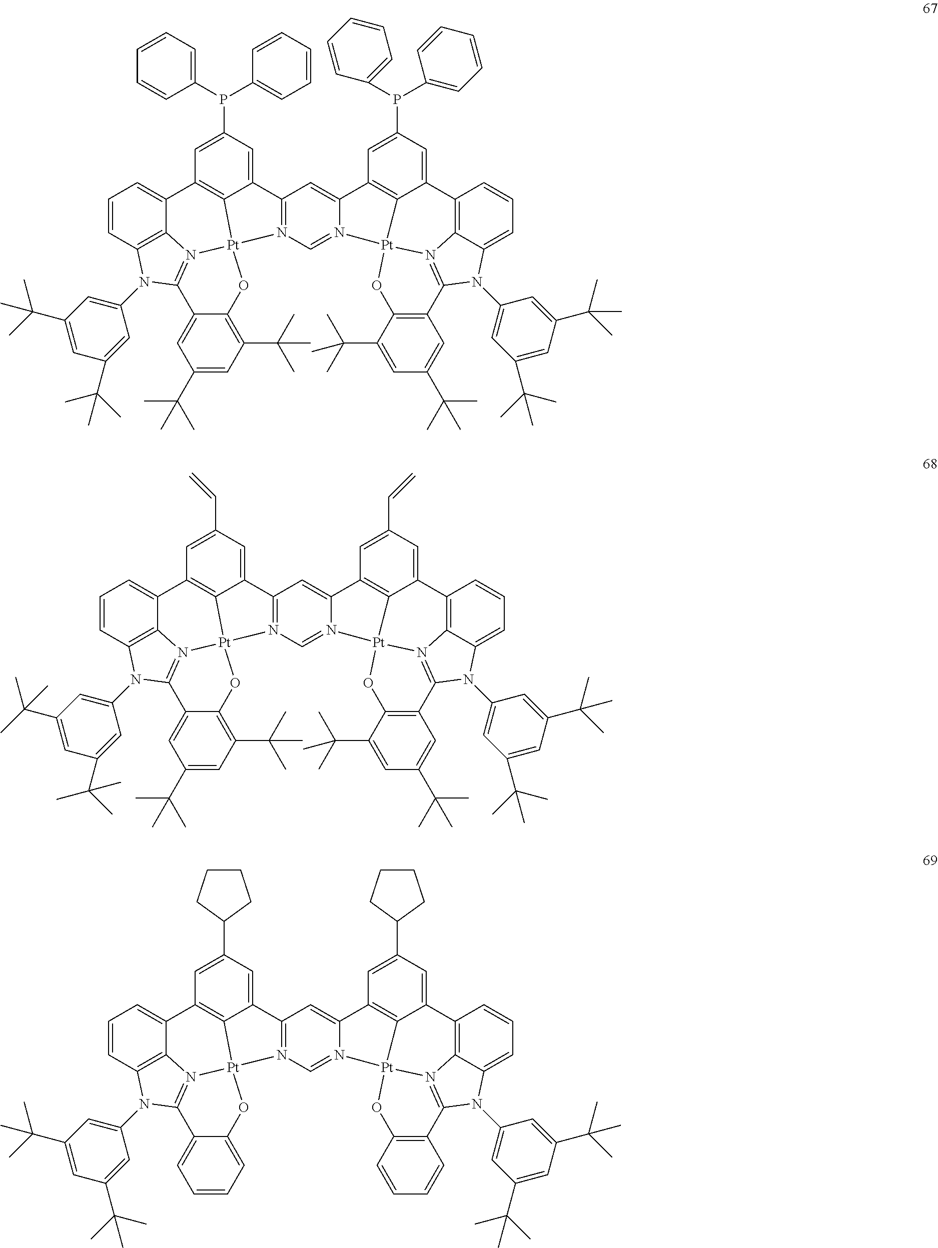

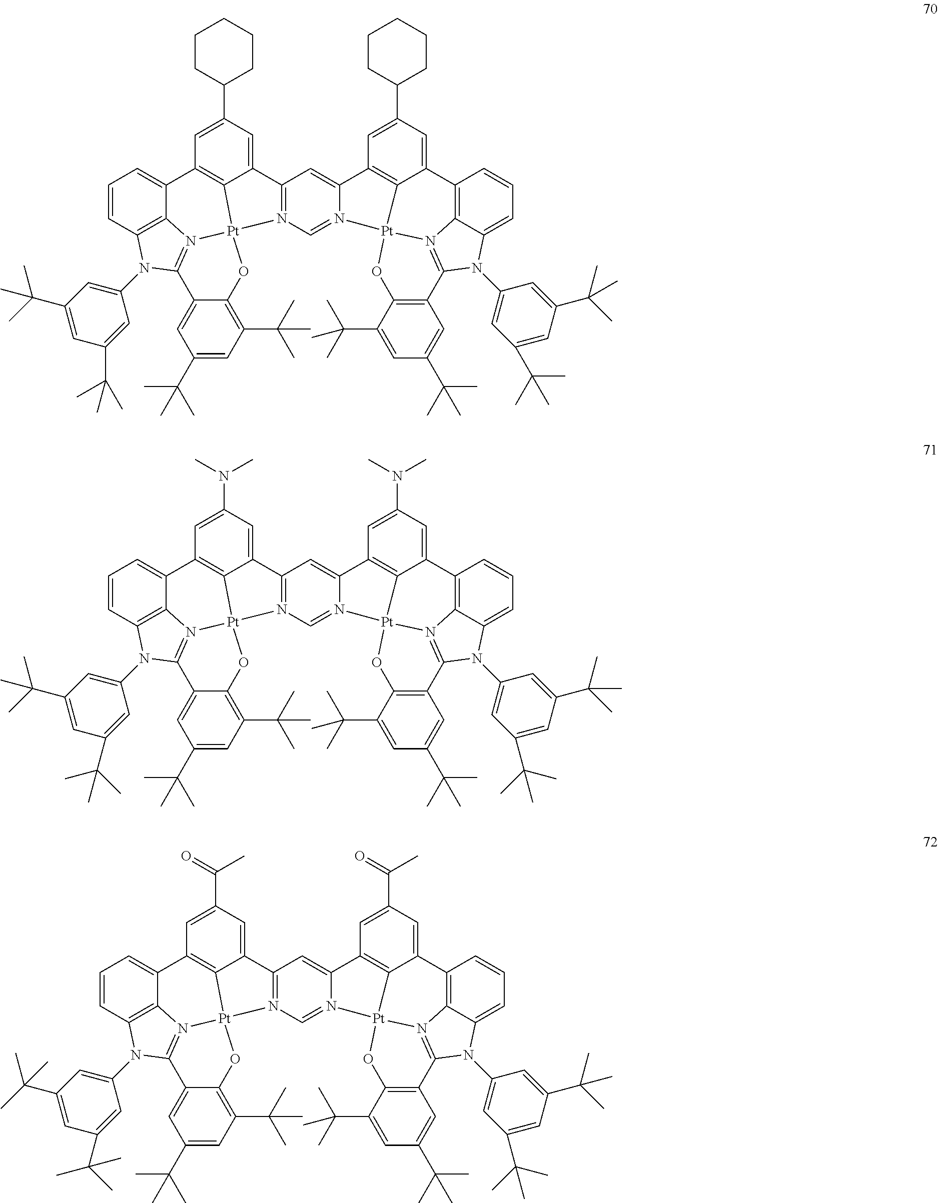

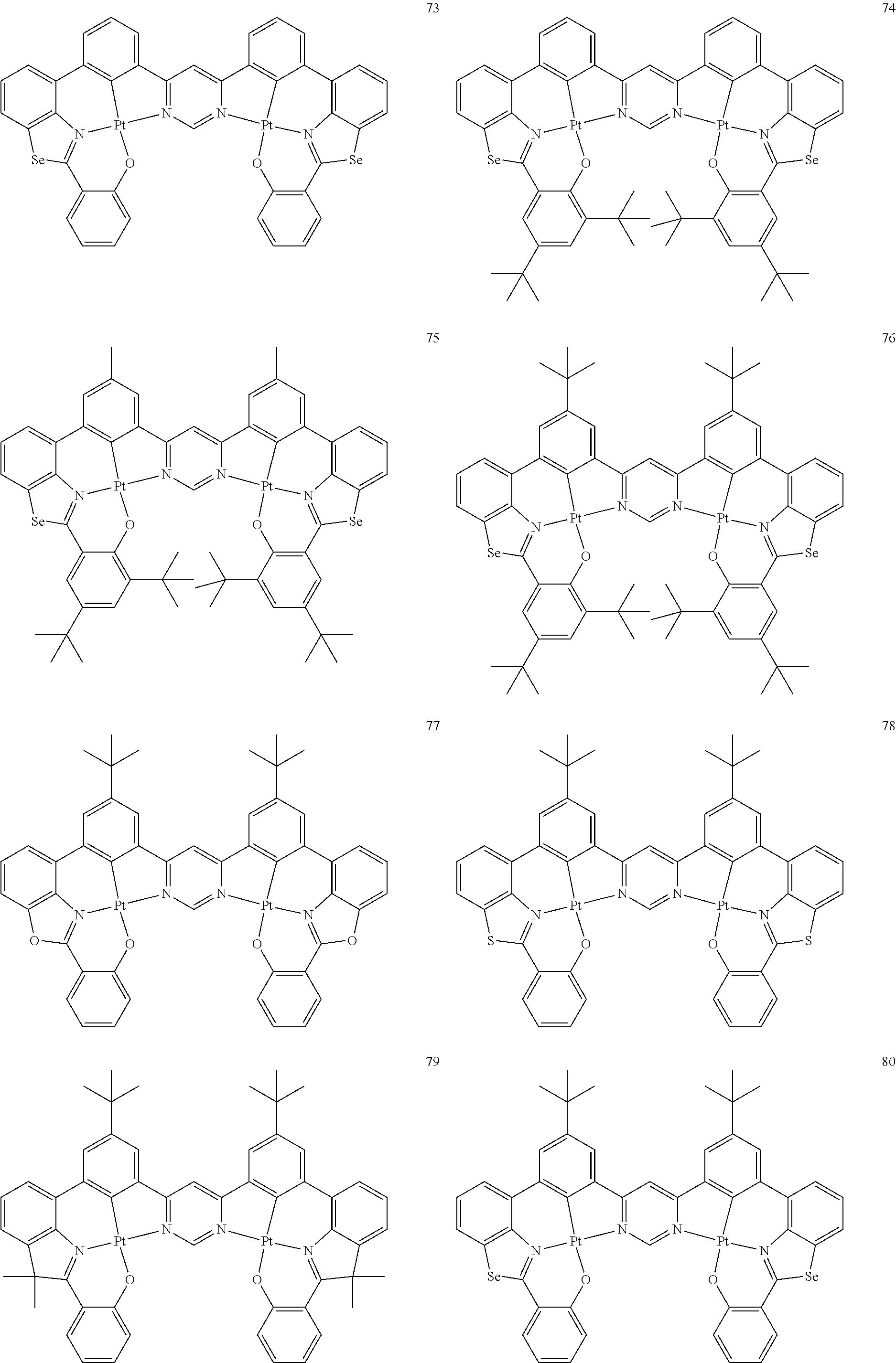

A ligand, being a precursor of the metal complex described above, has the following structural formula:

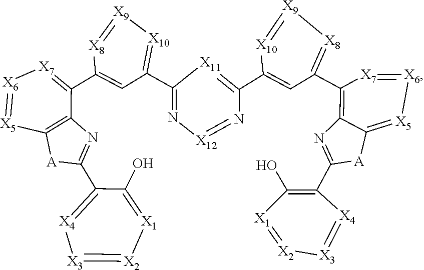

wherein X₁ to X₁₂ and A are defined above.

The present disclosure further provides use of the above-mentioned platinum complex in organic optoelectronic devices. The optoelectronic devices include, but are not limited to, organic light-emitting diodes, organic thin film transistors, organic photovoltaic devices, light-emitting electrochemical cells and chemical sensors, and in some embodiment are organic light-emitting diodes.

An organic light-emitting diode including the above-mentioned platinum complex, wherein the platinum complex is a luminescent material in a light-emitting device.

The organic light-emitting diode in the present disclosure includes a cathode, an anode, and an organic layer. The organic layer is one or more layers selected from a hole injection layer, a hole transport layer, a light-emitting layer, a hole blocking layer, an electron injection layer, or an electron transport layer, wherein these organic layers are not necessary to be all presented. At least one layer in the hole injection layer, the hole transport layer, the light-emitting layer, the hole blocking layer, the electron injection layer, and the electron transport layer includes the binuclear platinum complex represented by formula (I).

In an embodiment, the layer including the platinum complex represented by formula (I) is the light-emitting layer or the electron transport layer.

The total thickness of the organic layer of the device in the present disclosure is 1 nm to 1000 nm, in an embodiment is 1 nm to 500 nm, and in a further embodiment is 5 nm to 300 nm.

The organic layer can be formed into a film by vapor-deposition or solution processing.

The class of platinum complex luminescent materials disclosed in the present disclosure exhibit good luminescent performance, can be used as luminescent materials in organic light-emitting diodes, have good luminous efficiency and device lifetime, and thus possess the potential for application in the field of organic electroluminescent devices.

### BRIEF DESCRIPTION OF THE DRAWINGS

FIGURE is a structural view of an organic light-emitting diode device in the present disclosure;

wherein 10 represents a glass substrate, 20 represents an anode, 30 represents a hole injection layer, 40 represents a hole transport layer, 50 represents a light-emitting layer, 60 represents an electron transport layer, 70 represents an electron injection layer, 80 represents a cathode.

### DETAILED DESCRIPTION

The present disclosure will be further described in detail below with reference to examples.

### Example 1

### Synthesis of Complex 4

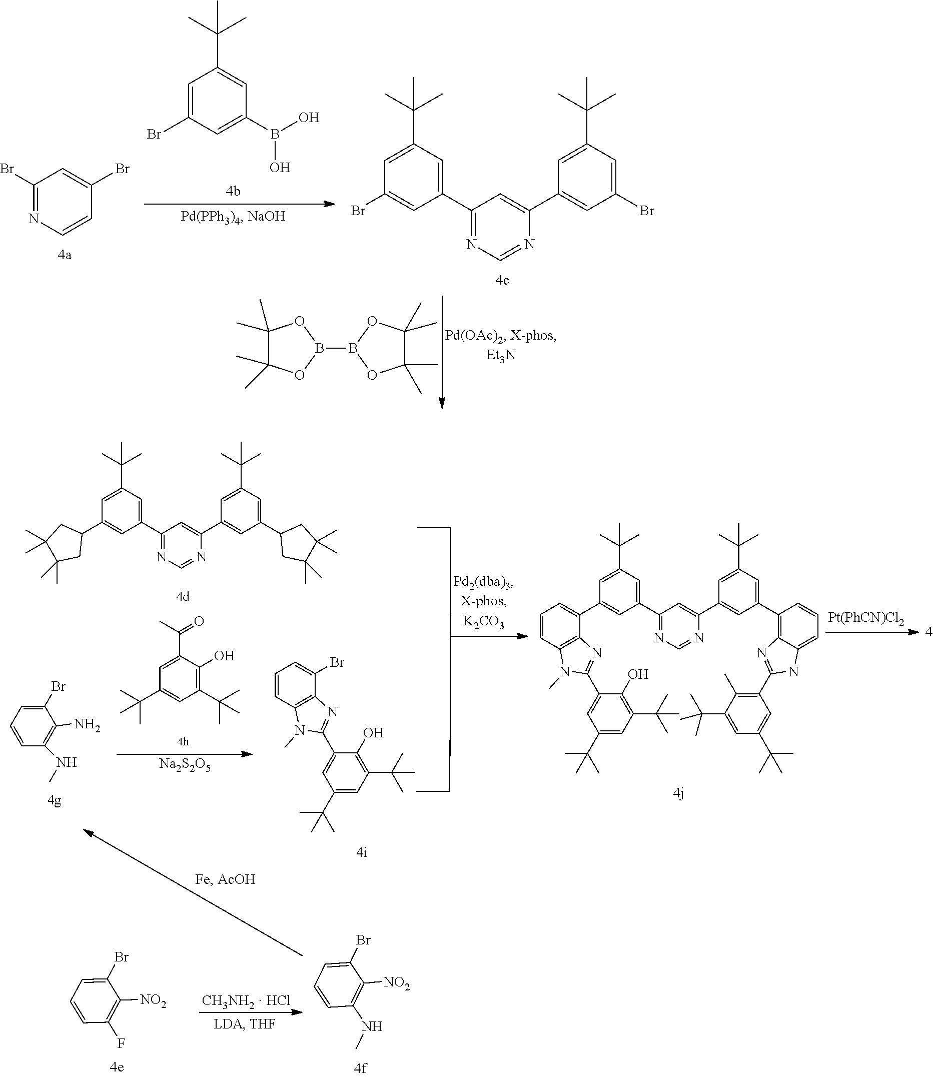

### Synthesis of Compound 4c

Under nitrogen gas protection, compound 4a (2.0 g, 8.4 mmol), compound 4b (4.53 g, 17.6 mmol), Pd(PPh₃)₄ (0.49 g, 0.42 mmol), NaOH (0.71 g, 17.6 mmol), toluene (40 mL) and water (10 mL) were added to a three-necked flask, then heated to 70° C., and stirred for 5 hours. When the reaction was completed, the reaction solution was cooled to room temperature, water (120 mL) was added, and the mixture was extracted with ethyl acetate. The organic phase was collected, and the solvent therein was removed under reduced pressure. The residue was subjected to silica gel column chromatography to obtain 2.50 g of a grayish white solid, with a yield of 59%. ESI-MS (m/z): 503.2 (M+1).

### Synthesis of Compound 4d

Under nitrogen gas protection, compound 4c (2 g, 3.98 mmol), bis(pinacolato)diboron (5.04 g, 19.8 mmol), Pd(OAc)₂ (88 mg, 0.4 mmol), X-phos (380 mg, 0.8 mmol), triethylamine (2.76 mL, 19.8 mmol) and toluene (40 mL) were added to a three-necked flask, then heated to 80° C. and reacted for 16 hours. When the reaction was completed, the reaction solution was cooled to room temperature, and filtered to remove the insoluble solid. The filtrate was extracted with ethyl acetate (200 mL) and water (200 mL) added. The organic phase was collected, and the solvent therein was removed under reduced pressure. The residue was subjected to silica gel column chromatography to obtain 1.2 g of a grayish white solid with a yield of 50.5%. ESI-MS (m/z): 597.5.2 (M+1).

### Synthesis of Compound 4f

Under argon gas protection, methylamine hydrochloride (1.2 g, 18.1 mmol) was dissolved in tetrahydrofuran (THF, 20 mL), lithium diisopropylamide (LDA, 15 ml, 2 mol/L) was added dropwise at 0° C., and the solution was reacted at 0° C. for 0.5 hours. Compound 4e (4.0 g, 18.1 mmol) dissolved in THF (10 mL) was added dropwise. The reaction solution was reacted at 0° C. for another 0.5 hours, and then heated to 45° C. and reacted for 16 hours. When the reaction was completed, water (50 mL) was added, and the mixture was extracted with ethyl acetate. The organic phase was collected, and the solvent therein was removed under reduced pressure. The residue was subjected to silica gel column chromatography to obtain a grayish white solid, with a yield of 45%. ESI-MS (m/z): 231.2 (M+1).

### Synthesis of Compound 4g

Under nitrogen gas protection, compound 4f (4.1 g, 17.8 mmol) and iron powder (4.13 g, 74.01 mmol) were added to acetic acid (50 mL), and reacted at 80° C. for 1 hour. When the reaction was completed, the reaction solution was filtered to remove the solid. The filtrate was washed with dichloromethane, and water (250 mL) was added. The mixture was extracted with dichloromethane, the organic phase was collected, and the solvent in the organic phase was removed under reduced pressure. The residue was subjected to silica gel column chromatography to obtain a grayish white solid, with a yield of 62%. ESI-MS (m/z): 201.2 (M+1).

### Synthesis of Compound 4i

Under nitrogen gas protection, compound 4 g (2.0 g, 10.0 mmol), compound 4h (3.2 g, 12.9 mmol) and Na₂S₂O₅ (5.6 g, 29.8 mmol) were dissolved in DMF (10 mL), and reacted at 90° C. for 24 hours. After completion of the reaction, water (100 mL) was added, and the mixture was extracted with dichloromethane. The organic phase was collected, and the solvent therein was removed under reduced pressure. The residue was subjected to silica gel column chromatography to obtain a grayish white solid, with a yield of 75%. ESI-MS (m/z): 415.2 (M+1).

### Synthesis of Compound 4j

Under nitrogen gas protection, compound 4d (1.5 g, 2.5 mmol), compound 4i (2.3 g, 5.5 mmol), Pd₂(dba)₃ (54 mg, 0.0585 mmol), X-phos (56 mg, 0.117 mmol), K₂CO₃ (0.71 g, 5.2 mmol) and toluene/ethanol/water (14 mL/3.5 mL/3.5 mL) were added to a three-necked flask, and reacted at 80° C. for 16 hours. The reaction solution was cooled to a room temperature, and then extracted with ethyl acetate (100 mL) and water (100 mL). The organic phase was collected, and the solvent therein was removed under reduced pressure. The residue was subjected to silica gel column chromatography to obtain 2.1 g of a yellow foamy solid, with a yield of 82%. ESI-MS (m/z): 1013.7 (M+1).

### Synthesis of Complex 4

Under nitrogen gas protection, compound 4j (0.5 g, 0.49 mmol), Pt(PhCN)₂Cl₂ (0.70 g, 1.48 mmol) and acetic acid (100 mL) were added to a three-necked flask, and reacted at 130° C. for 48 hours. When the reaction was completed, the reaction solution was cooled to room temperature, and water (800 mL) was added to precipitate a solid, which was collected by suction filtration. The solid was washed with water and dissolved in dichloromethane, and the solvent therein was removed under reduced pressure. The residue was subjected to silica gel column chromatography to obtain to obtain 0.41 g of a red solid, with a yield of 59%. ESI-MS (m/z): 1399.5 (M+1).

### Example 2

### Synthesis of Complex 16

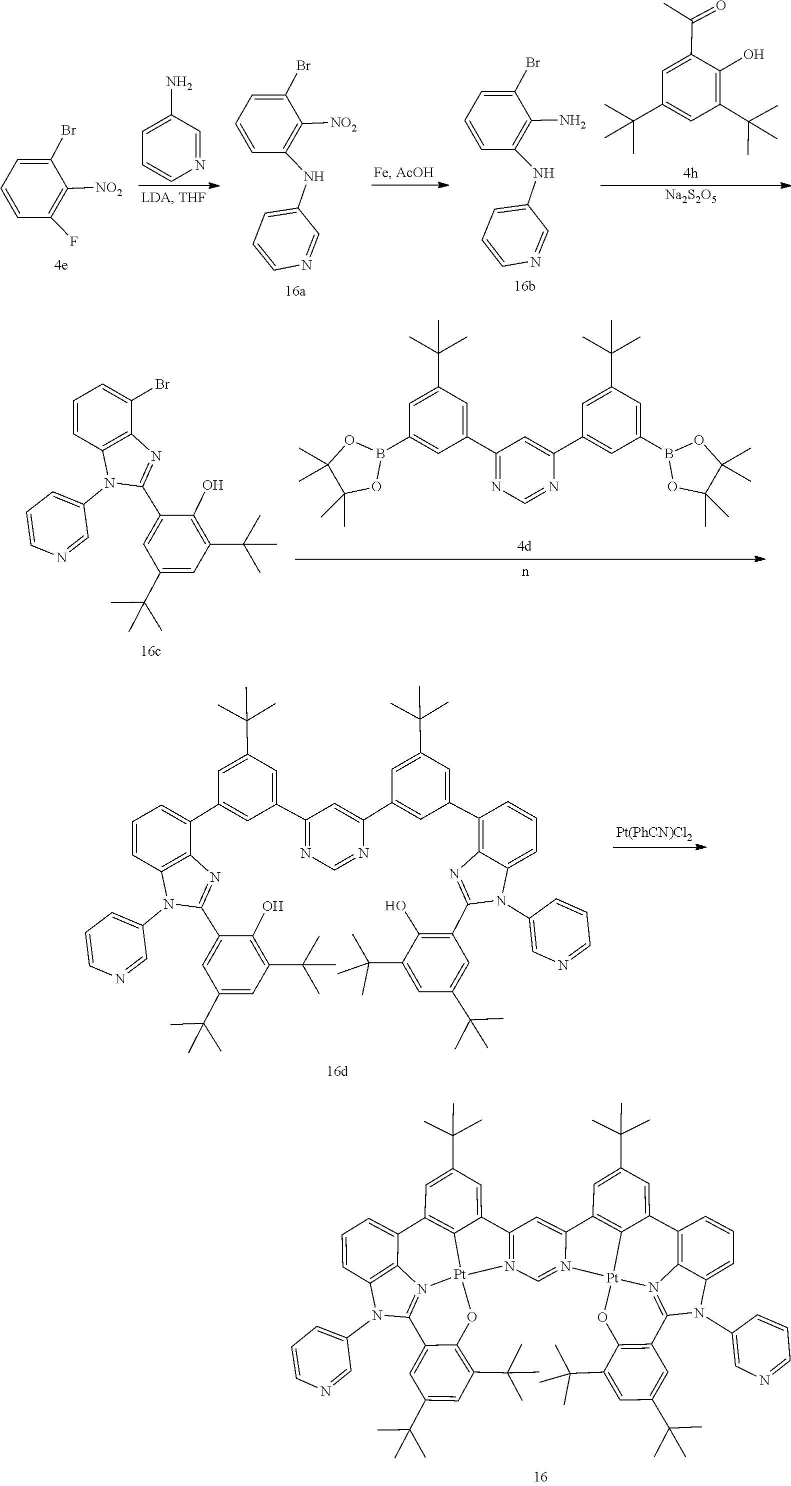

### Synthesis of Compound 16a

Compound 16a was prepared according to the synthesis of compound 4f, using 3-aminopyridine instead of methylamine hydrochloride, to obtain 6.3 g of a reddish brown solid with a yield of 47%. ESI-MS (m/z): 294.1 (M+1).

### Synthesis of Compound 16b

Compound 16b was prepared according to the synthesis of compound 4g, using compound 16a instead of compound 4f, to obtain 1.7 g of a reddish brown solid with a yield of 60%. ESI-MS (m/z): 264.2 (M+1).

### Synthesis of Compound 16c

Compound 16c was prepared according to the synthesis of compound 4f, using compound 16b instead of compound 4g, to obtain 2.1 g of a creamy white solid with a yield of 68%. ESI-MS (m/z): 478.2 (M+1).

### Synthesis of Compound 16d

Compound 16d was prepared according to the synthesis of compound 4j, using compound 16c instead of compound 4i, to obtain 3.6 g of a light yellow solid with a yield of 75%. ESI-MS (m/z): 1139.8 (M+1).

### Synthesis of Complex 16

Complex 16 was prepared according to the synthesis of complex 4, using compound 16d instead of compound 4j, to obtain 0.9 g of a red solid with a yield of 39%. ESI-MS (m/z): 1526.7 (M+1).

### Example 3

### Synthesis of Complex 20

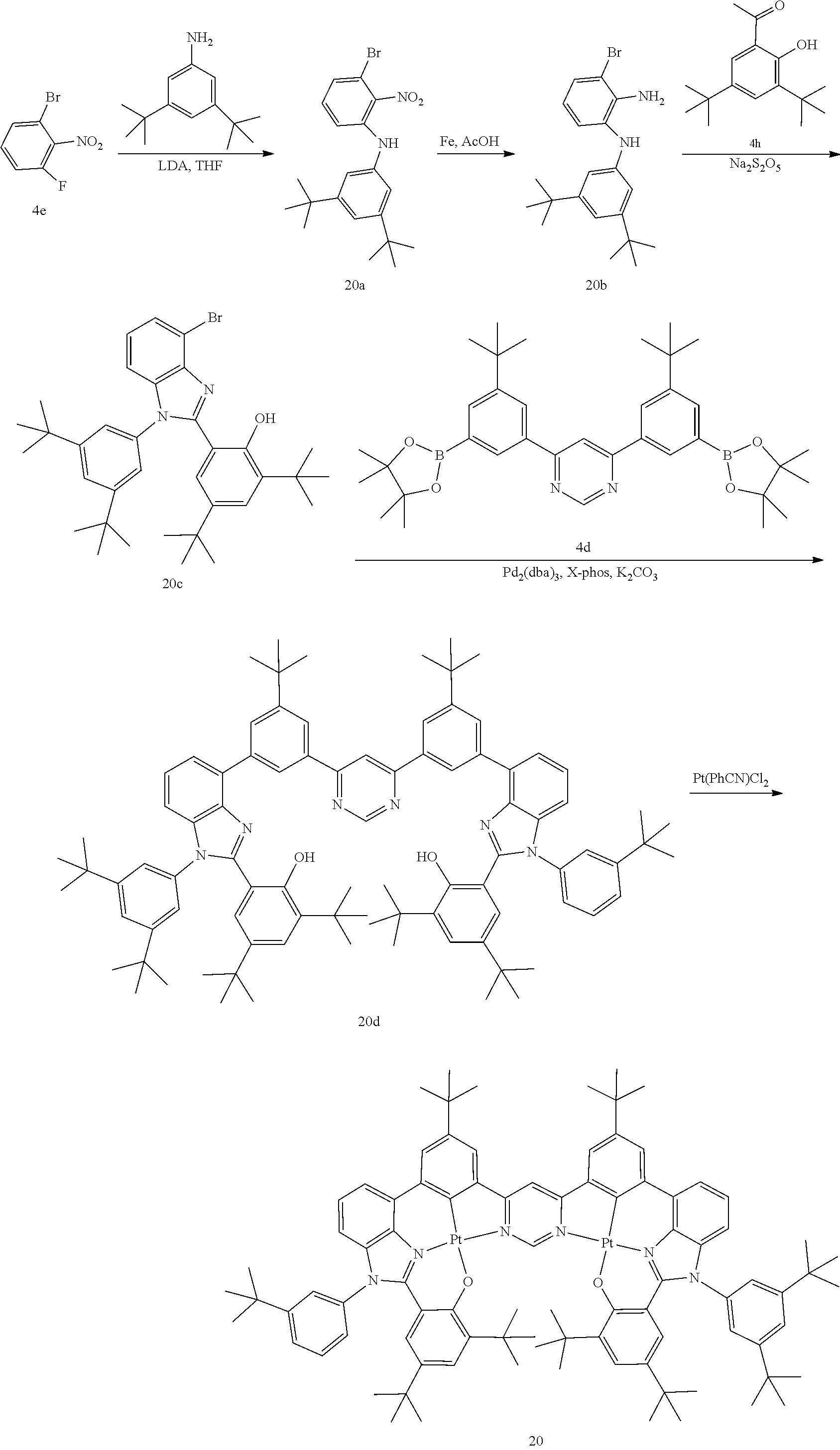

### Synthesis of Compound 20a

Compound 20a was prepared according to the synthesis of compound 4f, using 3,5-di-tert-butylaniline instead of methylamine hydrochloride, to obtain 7.0 g of a reddish brown solid with a yield of 50%. ESI-MS (m/z): 405.2 (M+1).

### Synthesis of Compound 20b

Compound 20b was prepared according to the synthesis of compound 4g, using compound 20a instead of compound 4f, to obtain 1.3 g of a reddish brown solid with a yield of 68%. ESI-MS (m/z): 375.2 (M+1).

### Synthesis of Compound 20c

Compound 20c was prepared according to the synthesis of compound 4f, using compound 20b instead of compound 4g, to obtain 1.2 g of a light yellow solid with a yield of 76%. ESI-MS (m/z): 589.3 (M+1).

### Synthesis of Compound 20d

Compound 20d was prepared according to the synthesis of compound 4j, using compound 20c instead of compound 4i, to obtain 2.6 g of light yellow solid with a yield of 68%. ESI-MS (m/z): 1363.0 (M+1).

### Synthesis of Complex 20

Complex 20 was prepared according to the synthesis of complex 4, using compound 20d instead of compound 4j, to obtain 1.3 g of red solid with a yield of 52%. ESI-MS (m/z): 1748.9 (M+1).

### Example 4

### Synthesis of Complex 77

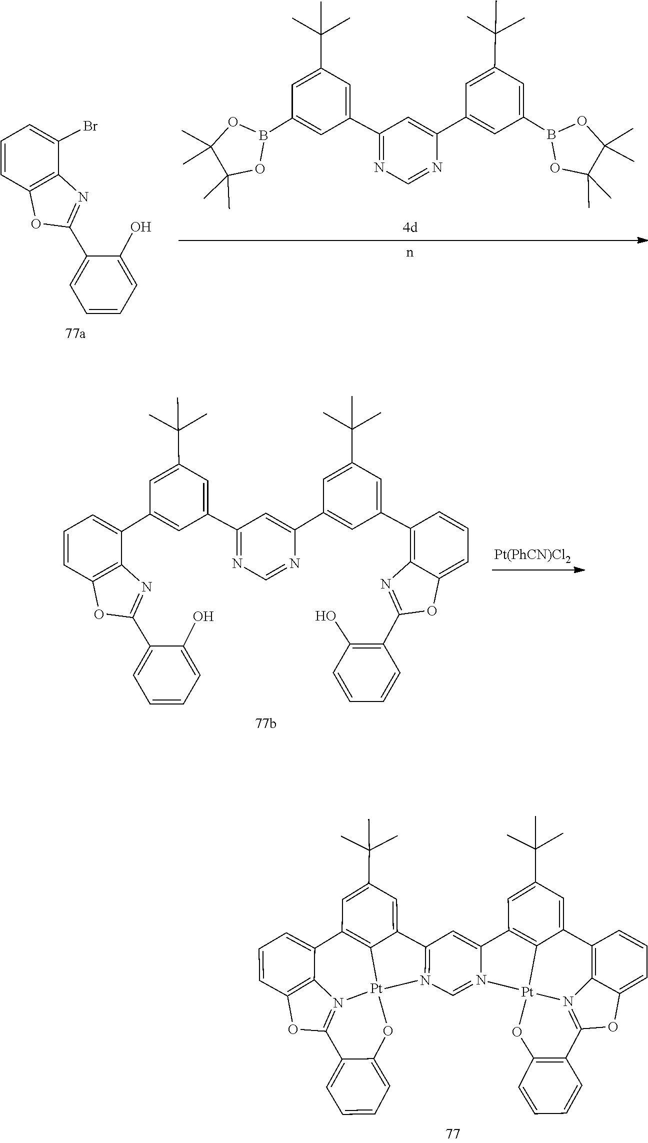

### Synthesis of Compound 77b

Compound 77b was prepared according to the synthesis of compound 4j, using compound 77a (the synthesis of which is referred to J. Mater. Chem. C, 2015, 3, 8212-8218) instead of compound 4i, to obtain 1.5 g of a light yellow solid with a yield of 53%. ESI-MS (m/z): 760.3 (M+1).

### Synthesis of Complex 77

Complex 77 was prepared according to the synthesis of complex 4, using compound 77b instead of compound 4j, to obtain 0.90 g of a red solid with a yield of 65%. ESI-MS (m/z): 1149.3 (M+1).

### Example 5

### Synthesis of Complex 78

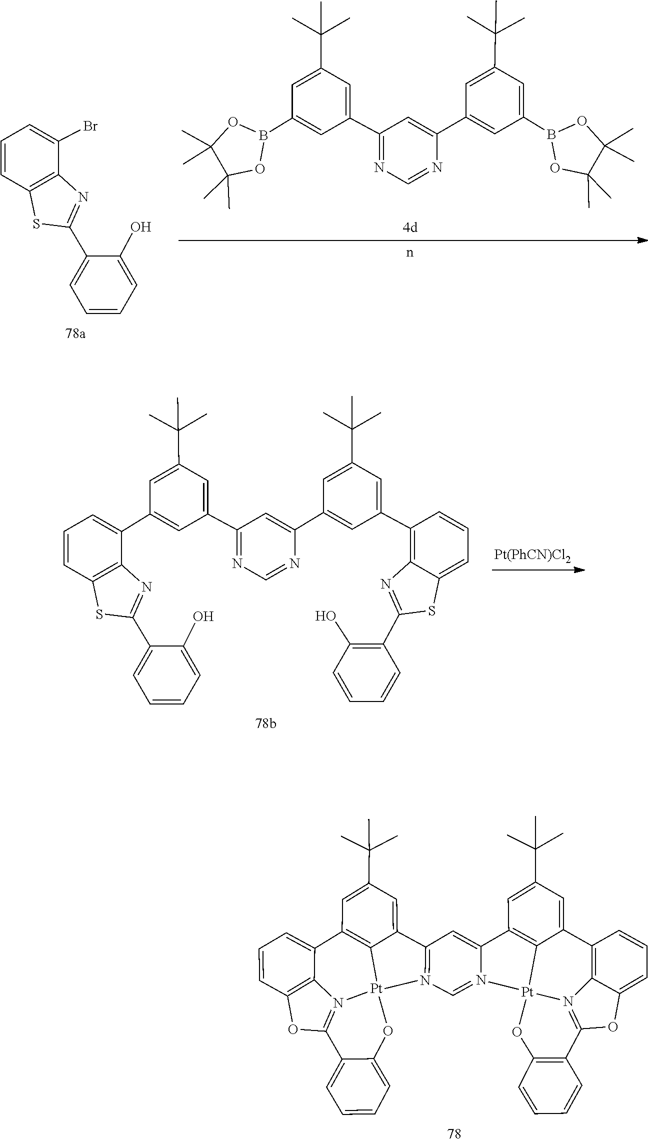

### Synthesis of Compound 78b

Compound 78b was prepared according to the synthesis of compound 4j, using compound 78a (the synthesis of which is referred to Tetrahedron, 2020, 76, 130982) instead of compound 4i, to obtain 1.7 g of a light yellow solid with a yield of 60%. ESI-MS (m/z): 795.4 (M+1).

### Synthesis of Complex 78

Complex 78 was prepared according to the synthesis of complex 4, using compound 78b instead of compound 4j, to obtain 0.98 g of a red solid with a yield of 67%. ESI-MS (m/z): 1181.2 (M+1).

Those skilled in the art should understand that the preparation methods described above are only illustrative examples, which can be modified by the those skilled in the art to obtain other compound structures of the present disclosure.

### Examples 6 to 10

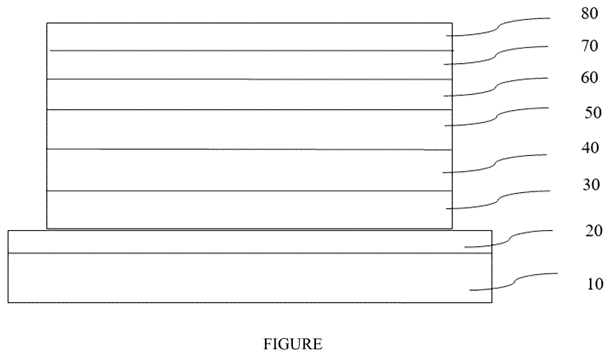

Organic light-emitting diodes were prepared by using the complex luminescent materials of the present disclosure. The structure of the device is shown in FIG. 1.

First, a transparent conductive indium tin oxide (ITO) glass substrate 10 with an anode 20 on top was washed with a detergent solution, deionized water, ethanol, acetone, and deionized water in sequence, and then treated with oxygen plasma for 30 seconds.

Then, a hole injection layer 30 was formed by spin-coating a solution of poly(3,4-ethylenedioxythiophene)-polystyrenesulfonate (PEDOT:PSS) on the ITO glass substrate.

Next, a hole transport layer 40 with a thickness of 10 nm was formed by spin-coating a polyvinylcarbazole (PVK) solution on the hole injection layer.

Subsequently, a light-emitting layer 50 with a thickness of 20 nm was formed by spin-coating a mixed toluene solution of a platinum complex and a bulk material on the hole transport layer. The mixed toluene solution includes the platinum complex, N,N′-bis(3-methylphenyl)-N,N′-diphenylbenzidine (TPD) and 2-(4-biphenyl)-5-(4-tert-butylphenyl)-1,3,4-oxadiazole (PBD) in a ratio of 6%:47%:47%. The platinum complexes in Examples 6 to 10 were complexes 4, 16, 20, 77 and 78 respectively.

Following that, 1,3,5-tris(1-phenyl-1H-benzimidazol-2-yl)benzene (TPBi) with a thickness of 40 nm as an electron transport layer 60 was deposited on the light-emitting layer by evaporation.

Finally, LiF with a thickness of 1 nm as an electron injection layer 70 and Al with a thickness of 100 nm as a cathode 80 were deposited by evaporation.

### Comparative Example 1

The device of Comparative Example 1 was prepared using the same preparation method with compound Ref-1 (synthesized with reference to Chem. Sci., 2021, 12, 6172-6180) instead of the platinum complex.

The structural formulas of PVKH, TBD, PBD, TPBi and Ref-1 in the device are as follows.

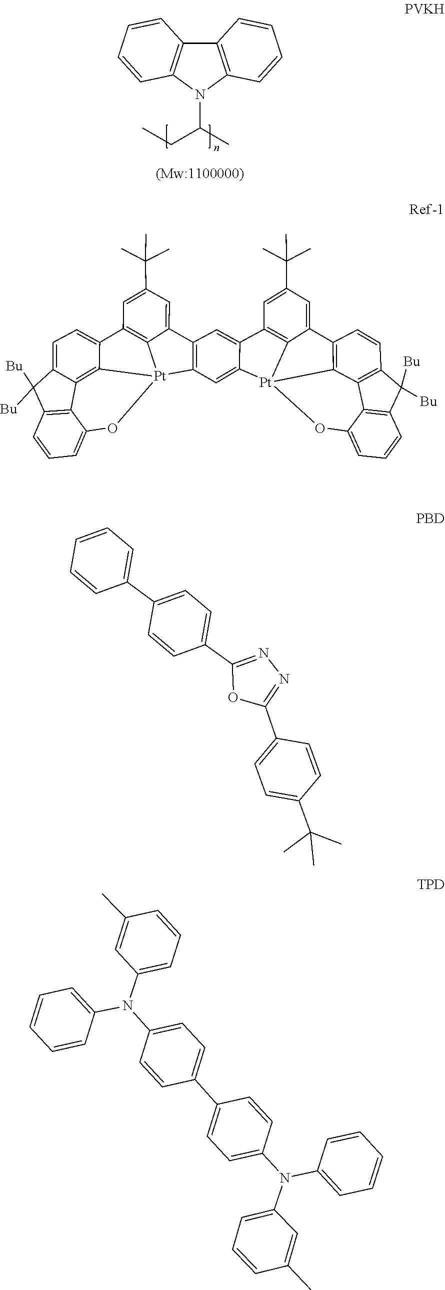

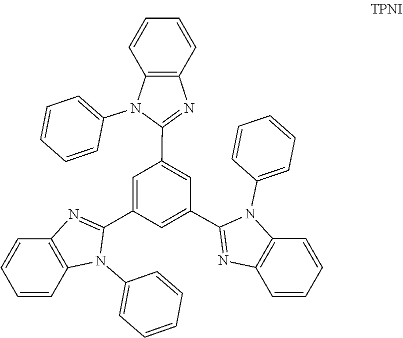 [SMILES: c1ccc(-n2c(-c3cc(-c4nc5ccccc5n4-c4ccccc4)cc(-c4nc5ccccc5n4-c4ccccc4)c3)nc3ccccc32)cc1]

The performances of the organic electroluminescent devices in Examples 6 to 10 at a current density of 10 mA/cm² are listed in the following table.

| | | | | | Device |
|---|---|---|---|---|---|
| | | Driving | Luminous | | Lifetime |
| Device No. | Complex | Voltage | Efficiency | Light Color | (LT90) |
| Comparative | Ref-1 | 1 | 1 | Red | 1 |
| Example 1 | | | | | |
| Example 6 | 4 | 0.98 | 1.12 | Dark red | 1.3 |
| Example 7 | 16 | 0.94 | 1.20 | Dark red | 1.8 |
| Example 8 | 20 | 0.96 | 1.25 | Dark red | 2.7 |
| Example 9 | 77 | 0.93 | 1.10 | Dark red | 2.1 |
| Example 10 | 78 | 0.90 | 1.10 | Dark red | 2.9 |

Note:
The device performance test took Comparative Example 1 as the benchmark, all indexes of which were set to 1. LT90 represents the time it takes for the device brightness to decay to 90% of its initial brightness, which was 1000 cd/m².

It can be seen from the data in Table 1 that, under the same conditions, the platinum complex materials of the present disclosure, when applied to the organic light-emitting diodes, emit dark red light and exhibit lower driving voltages and higher luminous efficiencies compared with the comparative molecule ref-1. It is to be noted that the device lifetimes of the organic light-emitting diodes based on the complexes of the present disclosure are significantly longer than that of the organic light-emitting diode based on the complex material in the comparative example, indicating good industrialization potential.

The various embodiments described above are only examples and are not intended to limit the scope of the present disclosure. Various materials and structures in the present disclosure can be replaced by other materials and structures without departing from the spirit of the present disclosure. It should be understood by those of ordinary skill in the art that various modifications and changes can be made according to the concept of the present disclosure without creative efforts. Therefore, technical solutions that can be obtained through analysis, reasoning or partial research on the basis of the existing technology should be within the protection scope defined by the appended claims.

1. A binuclear platinum complex being a compound having a structure of formula (I): 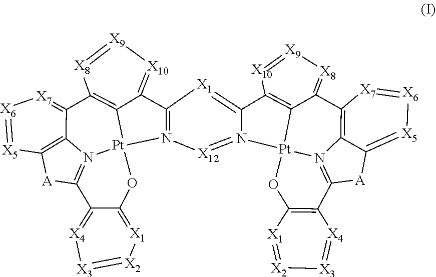
wherein:
X₁ to X₁₂ are each independently selected from N or CR;
A is selected from CR¹ R², NR³, O, S, or Se;
R, R¹, R², and R³ are each independently selected from the following groups: hydrogen, deuterium, halogen, amino group, C1-C20 alkylcarbonyl, carboxyl, aldehyde, C1-C20 thioalkyl, cyano group, sulfonyl, phosphino group, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted alkoxy having 1 to 20 carbon atoms, substituted or unsubstituted aryl having 6 to 30 carbon atoms, or substituted or unsubstituted heteroaryl having 3 to 30 carbon atoms; or any two adjacent R, R¹, R², and R³ are connected to form a ring;
the heteroatom in the heteroaryl group is one or more of N, S, or O;
the substitution is by halogen, amino group, cyano group, C6-C12 aryl, or C1-C4 alkyl.

2. The binuclear platinum complex according to claim 1, wherein R, R¹, R², and R³ are each independently selected from hydrogen, deuterium, halogen, amino group, C1-C6 thioalkyl, cyano group, substituted or unsubstituted alkyl having 1 to 6 carbon atoms, substituted or unsubstituted cycloalkyl having 3 to 6 ring carbon atoms, substituted or unsubstituted alkenyl having 2 to 6 carbon atoms, substituted or unsubstituted alkoxy having 1 to 6 carbon atoms, substituted or unsubstituted aryl having 6 to 12 carbon atoms, or substituted or unsubstituted heteroaryl having 3 to 6 carbon atoms.

3. The binuclear platinum complex according to claim 2, wherein R, R¹, R², and R³ are each independently selected from hydrogen, deuterium, halogen, cyano group, C1-C4 alkyl, substituted or unsubstituted cycloalkyl having 3 to 6 ring carbon atoms, substituted or unsubstituted aryl having 6 to 12 carbon atoms, or substituted or unsubstituted heteroaryl having 3 to 6 carbon atoms;
A is selected from NR³, O, or S;
the substitution is by phenyl or C1-C4 alkyl.

4. The binuclear platinum complex according to claim 3, wherein R, R¹, R², and R³ are each independently selected from hydrogen, deuterium, cyano group, methyl, isopropyl, isobutyl, tert-butyl, substituted or unsubstituted cyclopentyl, substituted or unsubstituted cyclohexyl, substituted or unsubstituted phenyl, substituted or unsubstituted pyridyl, substituted or unsubstituted pyrazinyl, or substituted or unsubstituted pyrimidinyl.

5. The binuclear platinum complex according to claim 3, wherein the formula (I) is the following structure:

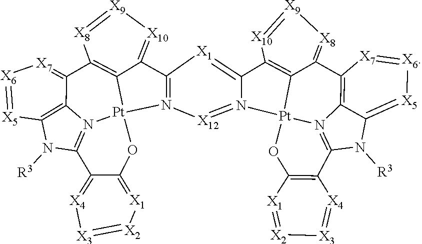

6. The binuclear platinum complex according to claim 5, wherein R and R³ are independently selected from hydrogen, deuterium, methyl, tert-butyl, substituted or unsubstituted cyclopentyl, substituted or unsubstituted cyclohexyl, substituted or unsubstituted phenyl, or substituted or unsubstituted pyridyl.

7. The binuclear platinum complex according to claim 6, wherein the formula (I) is the following structure:

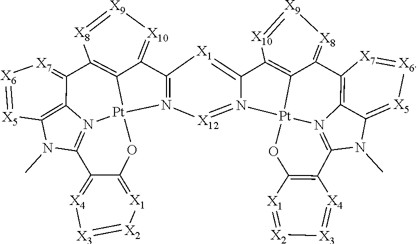

8. The binuclear platinum complex according to claim 1, wherein at most one of X₁ to X₄ is N, and the rest are CR; at most one of X₅ to X₇ is N, and the rest are CR; at most one of X₈ to X₁₀ is N, and the rest are CR; X₁₂ is CH, and X₁₁ is N or CH.

9. The binuclear platinum complex according to claim 8, wherein X₁, X₃, and X₉ are CR; X₂, X₄ to X₈, and X₁₀ are CH, R is hydrogen, deuterium, methyl, tert-butyl, cyclopentyl, cyclohexyl, phenyl, or pyridyl.

10. The binuclear platinum complex according to claim 1 being one of the following compounds:

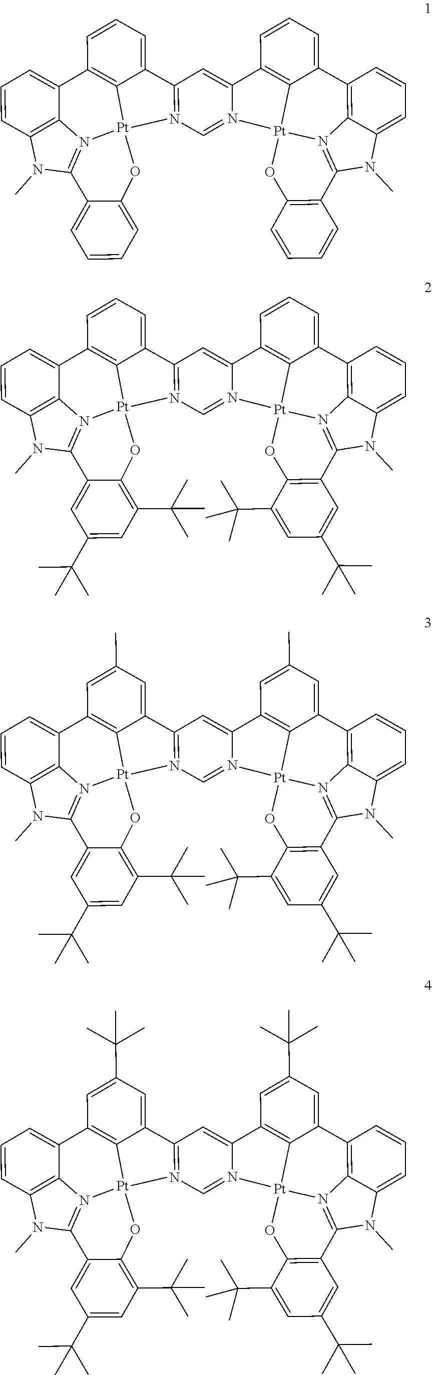

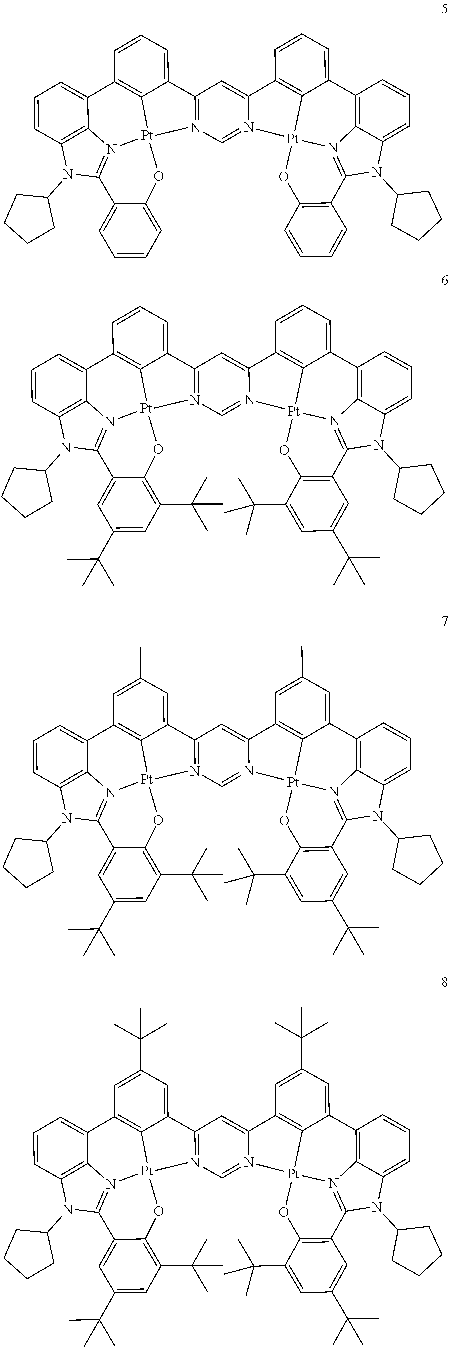

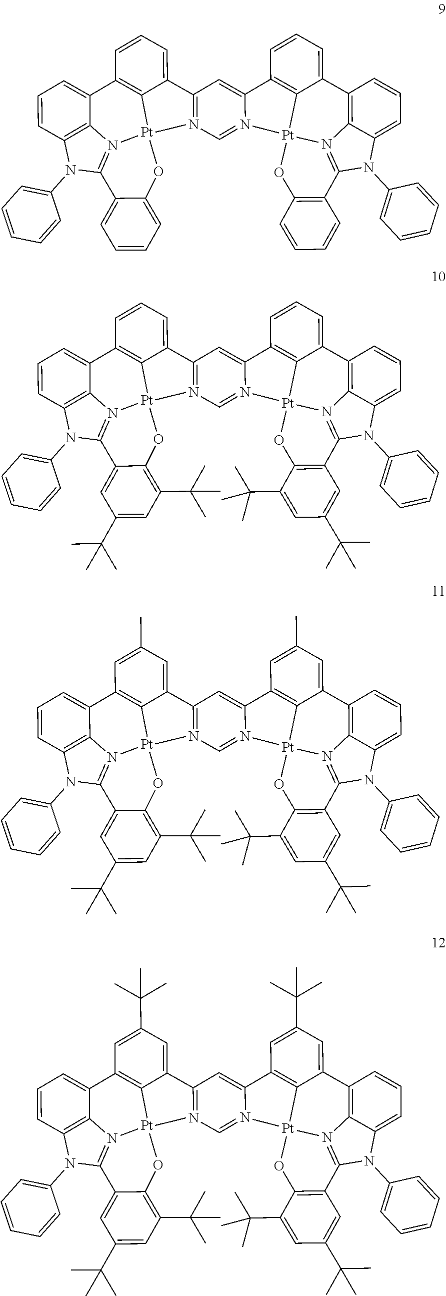

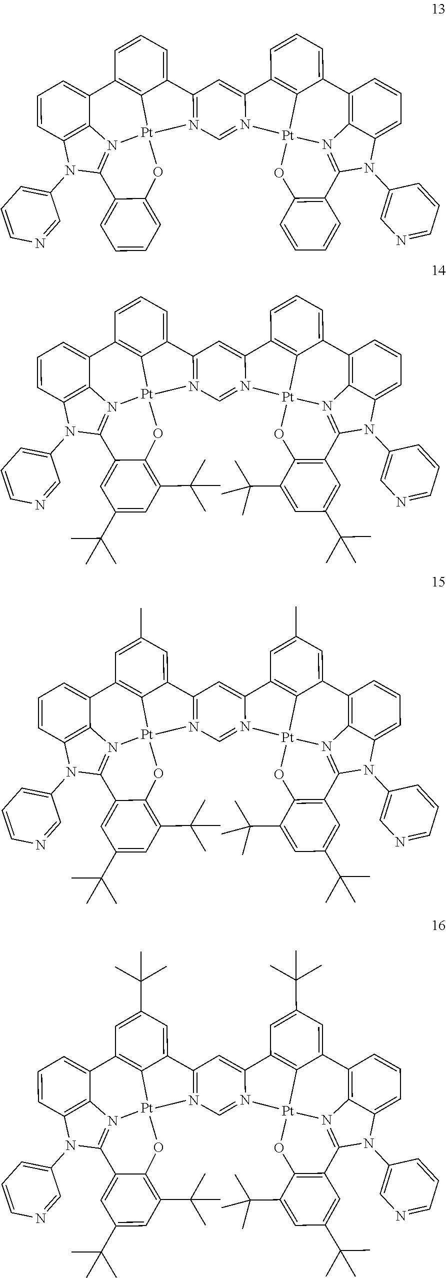

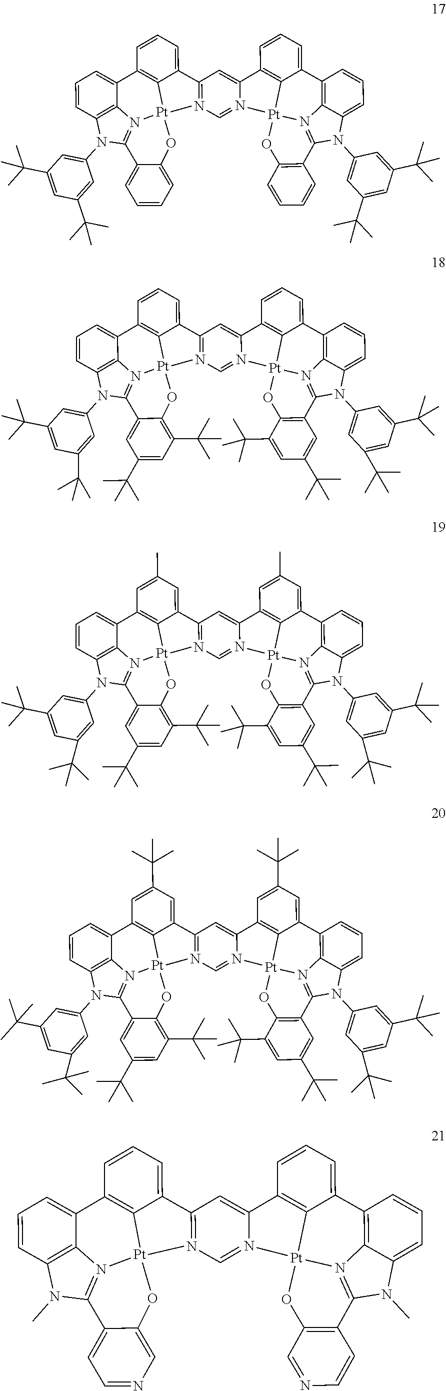

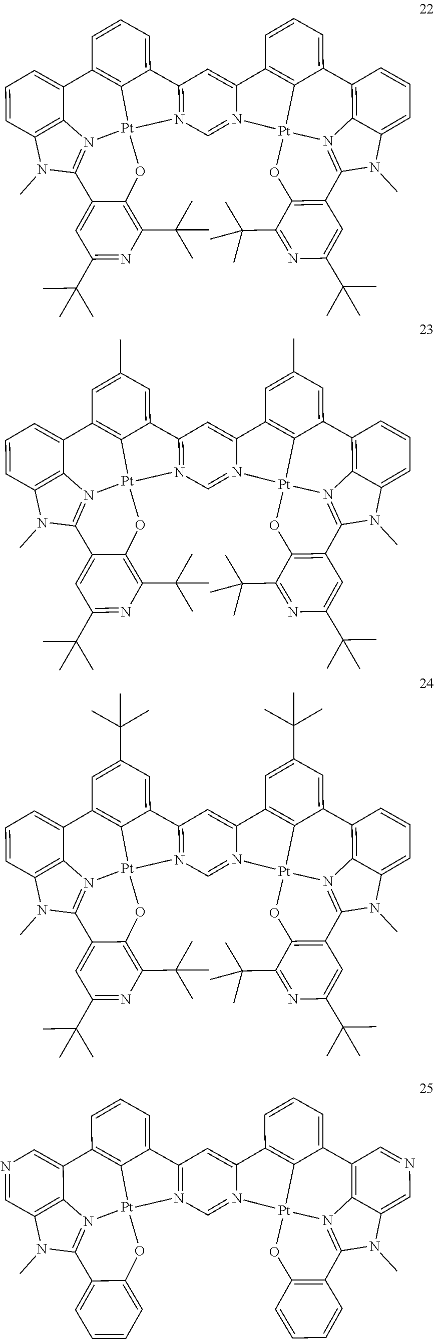

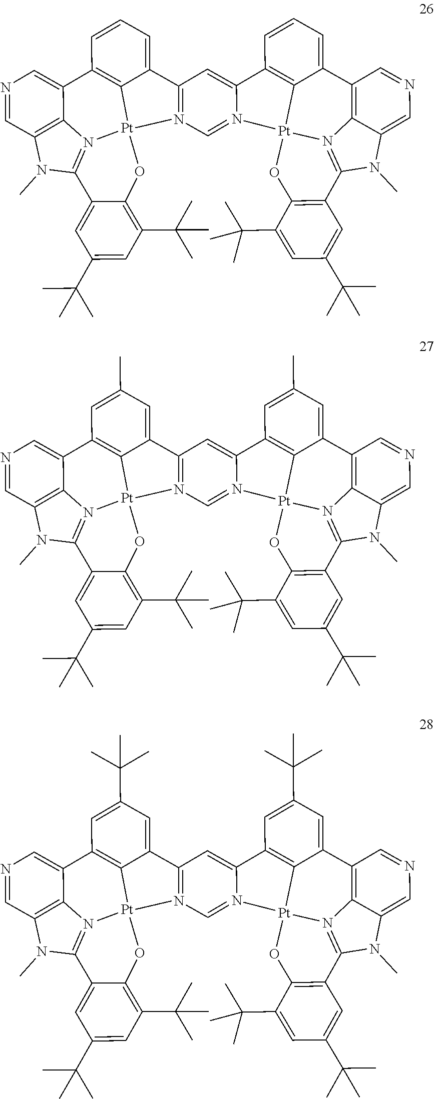

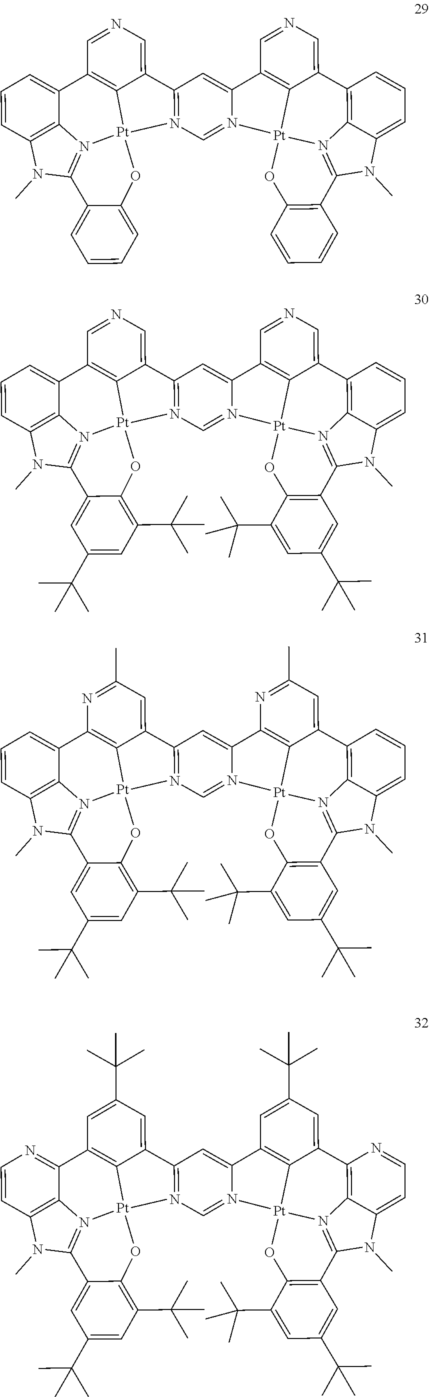

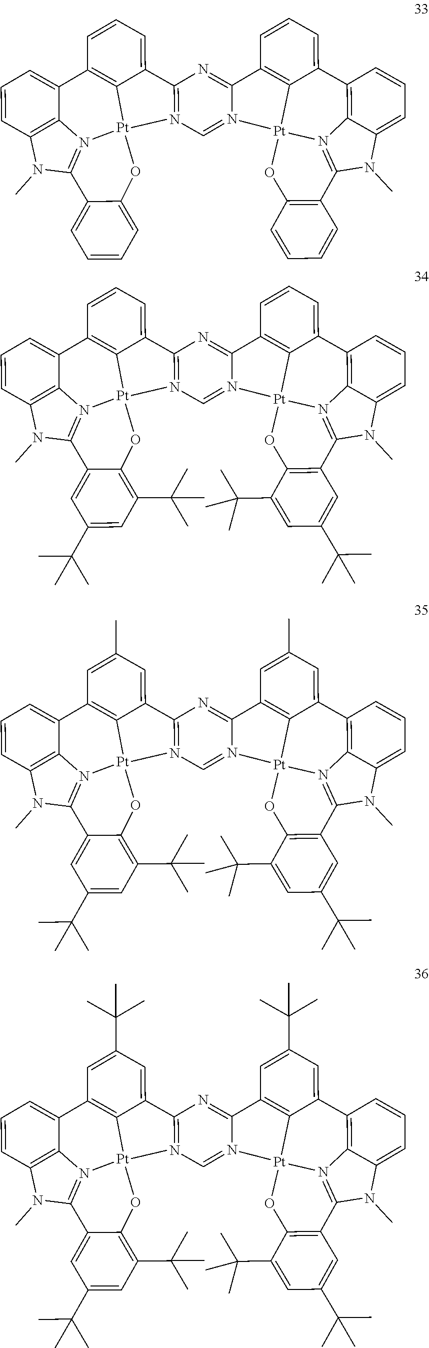

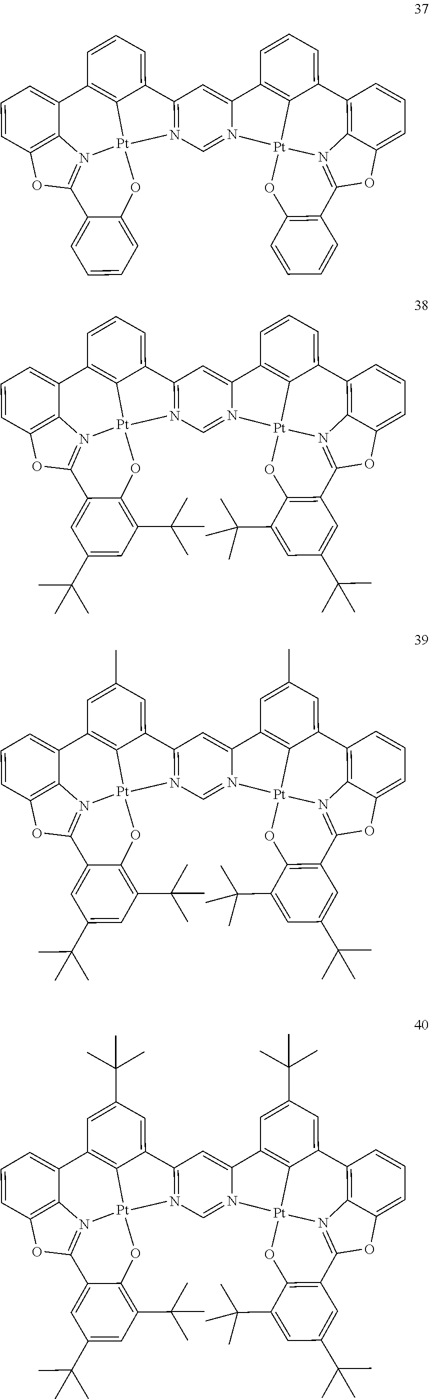

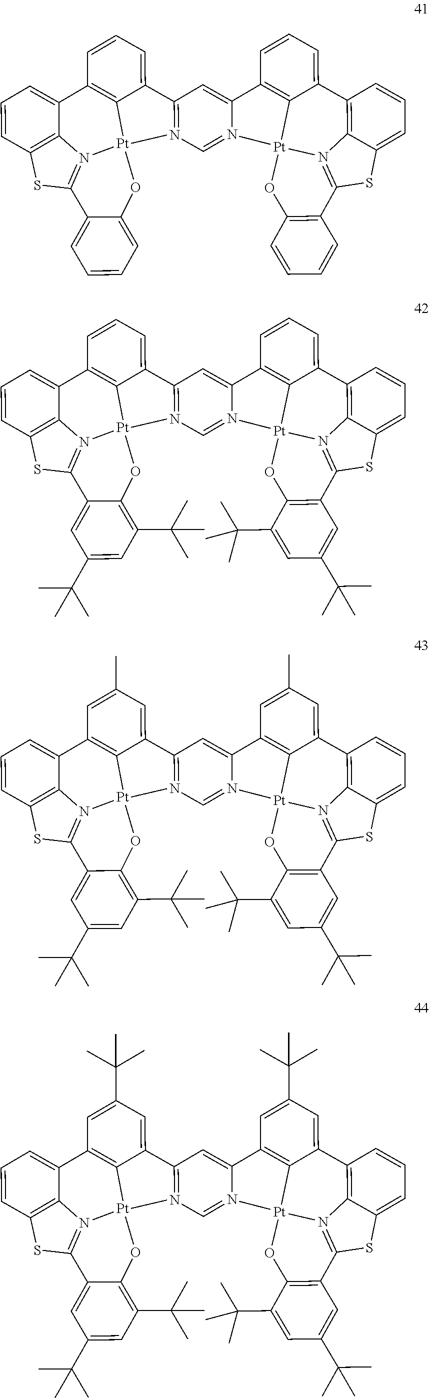

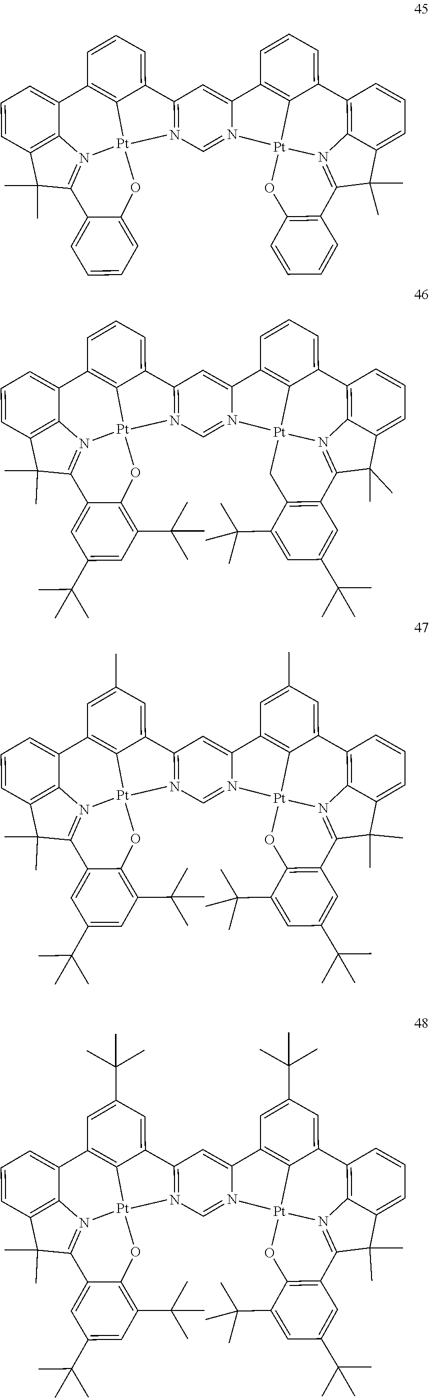

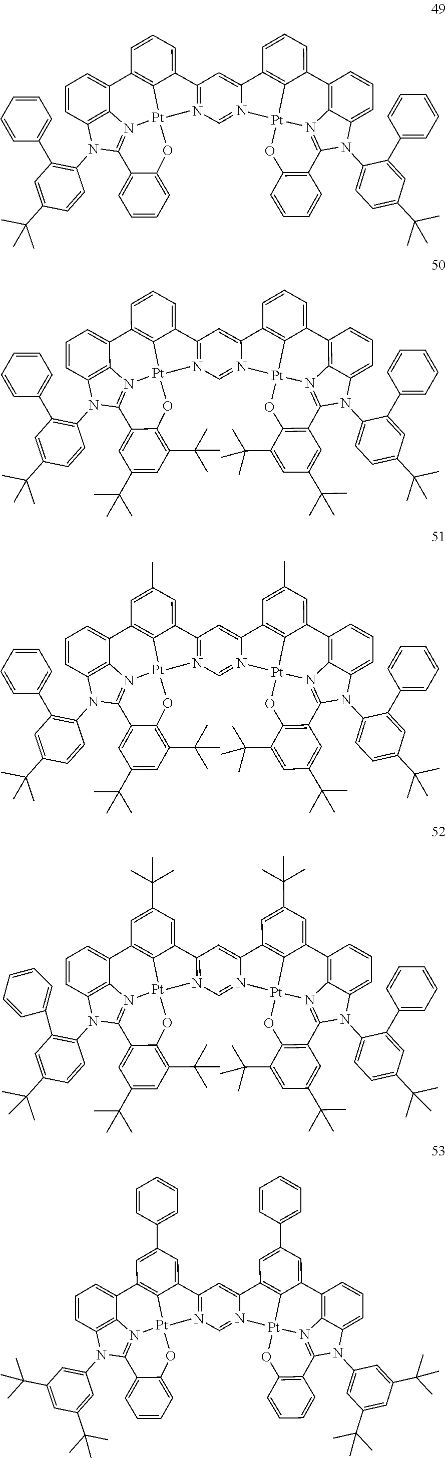

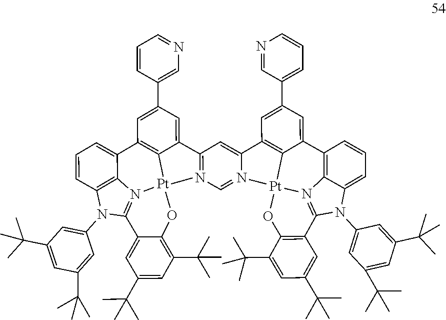

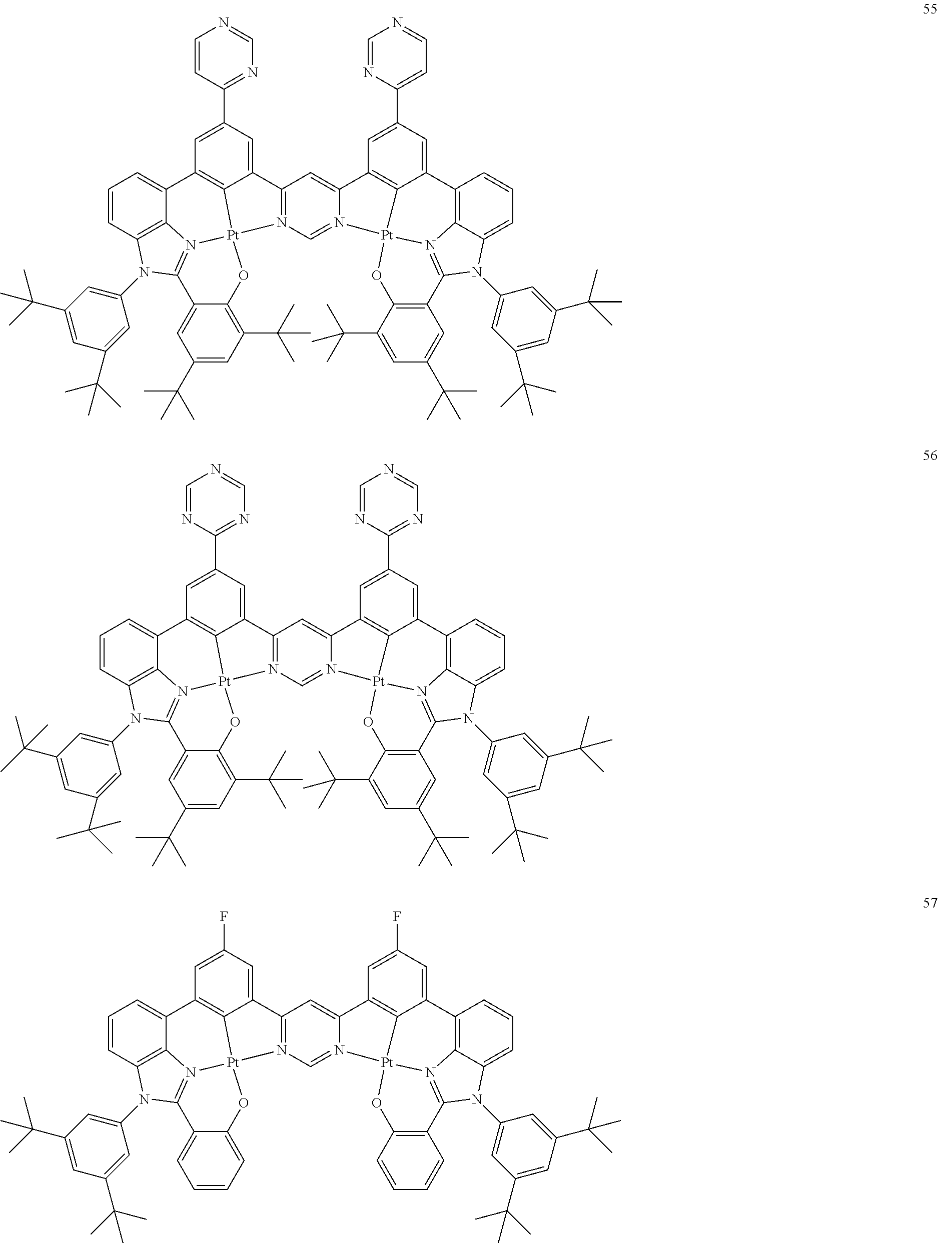

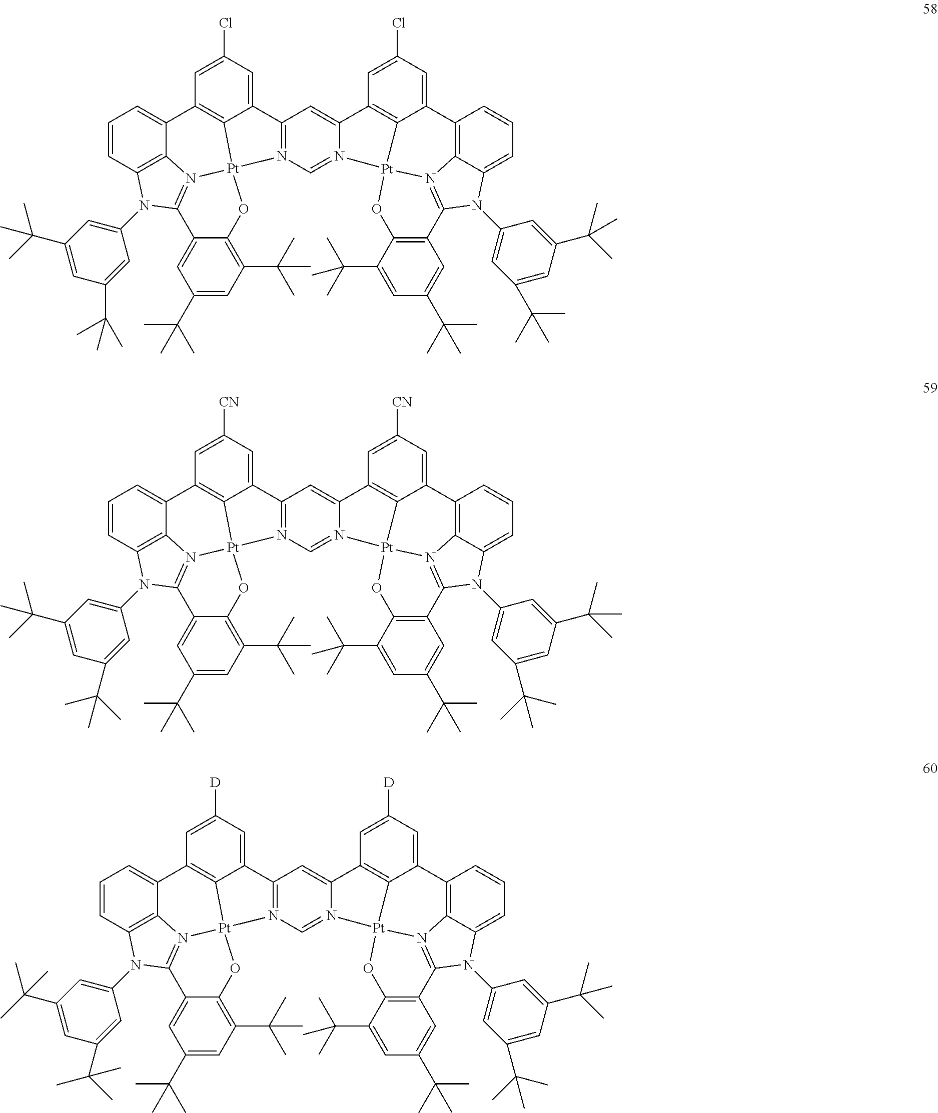

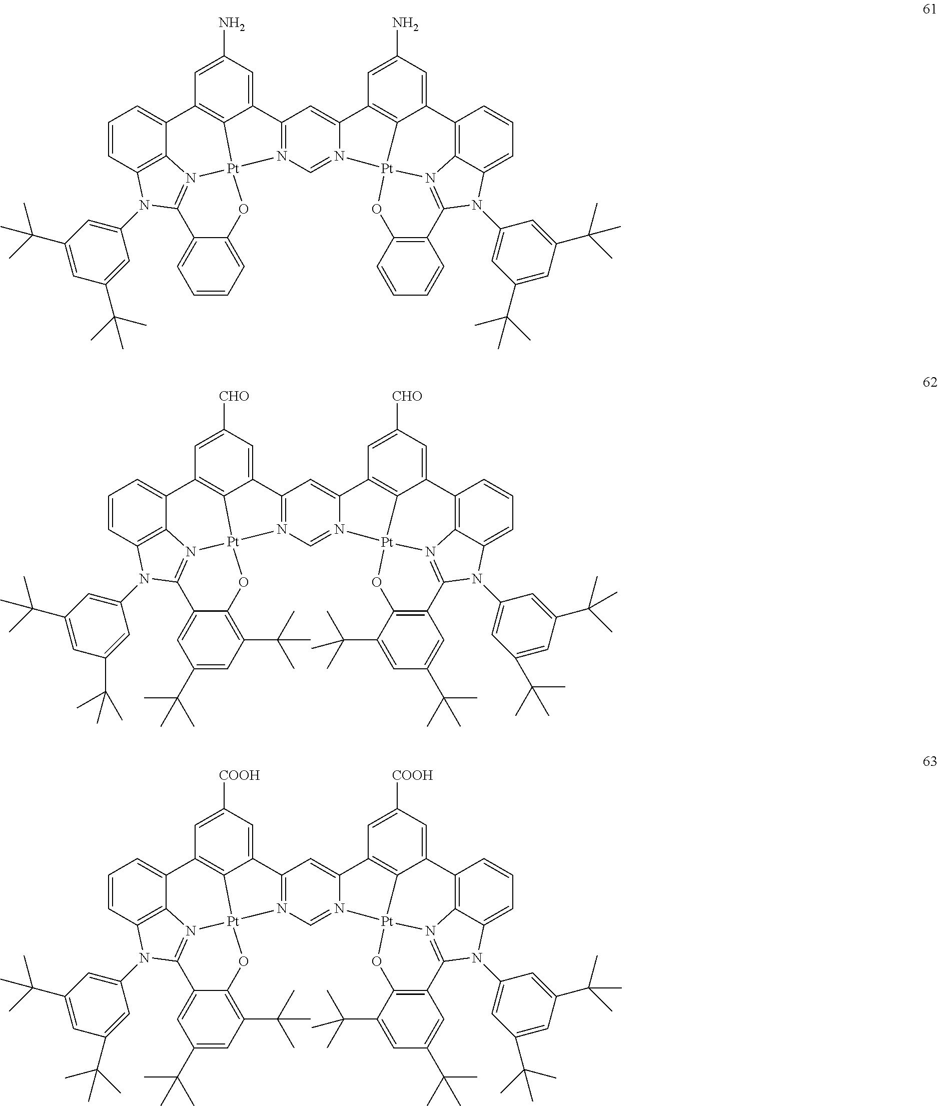

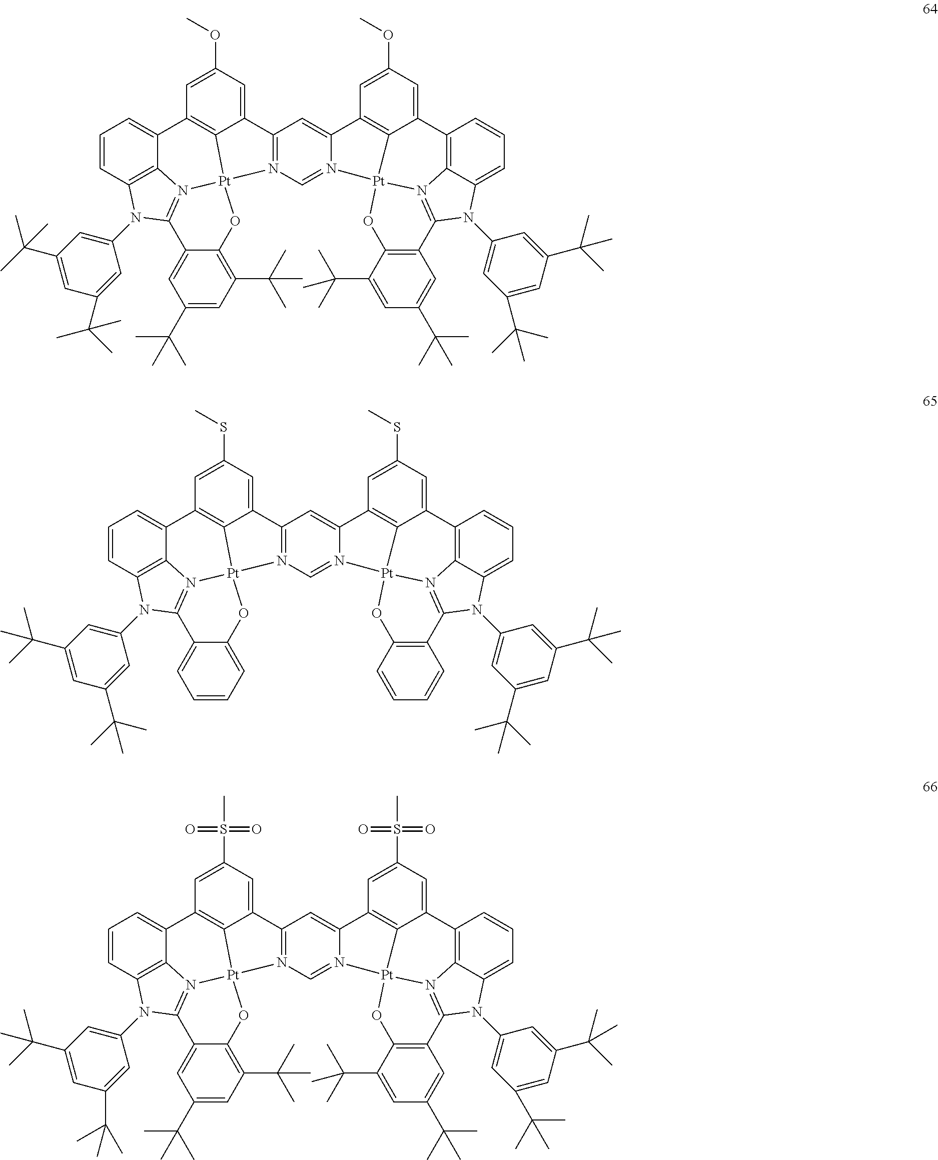

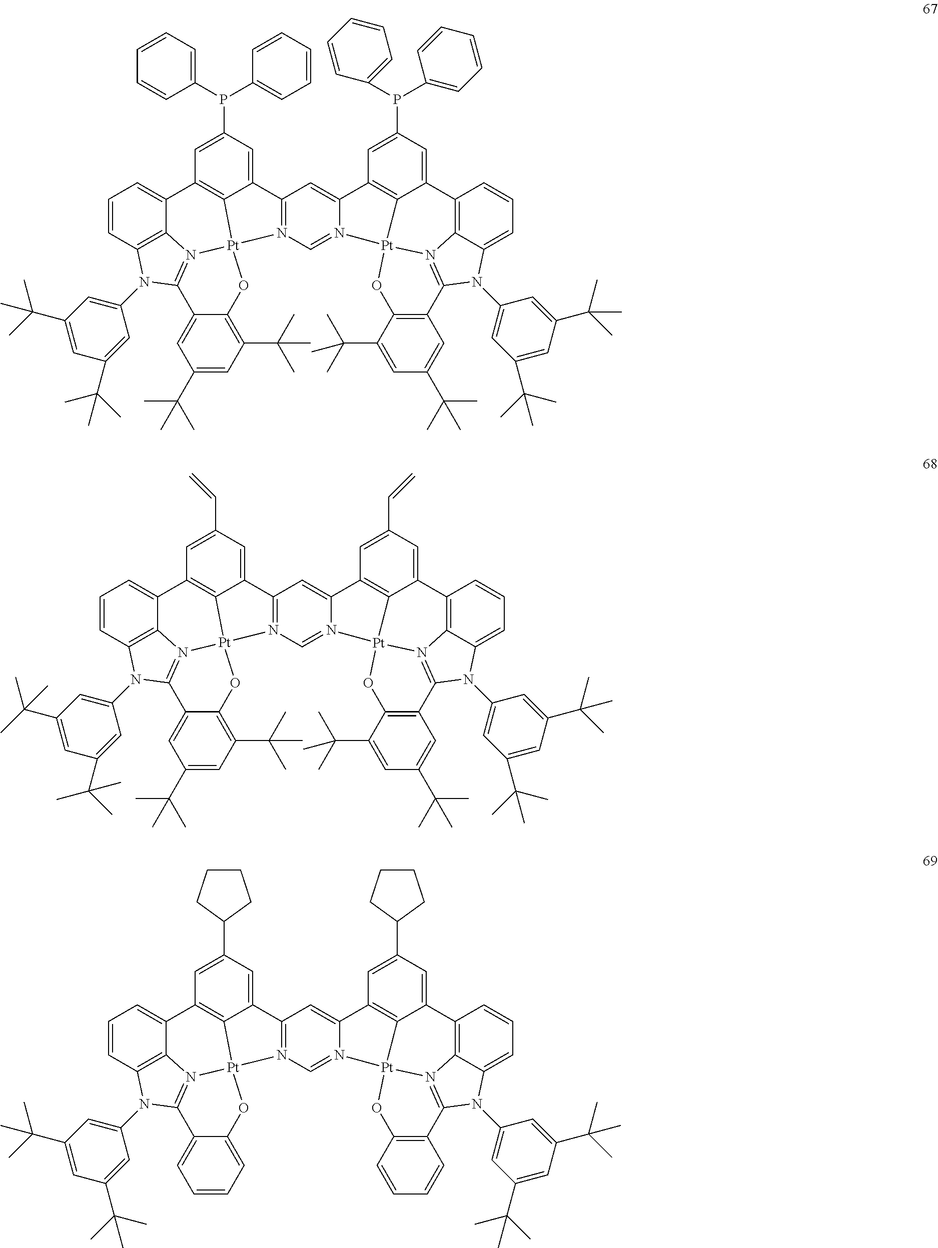

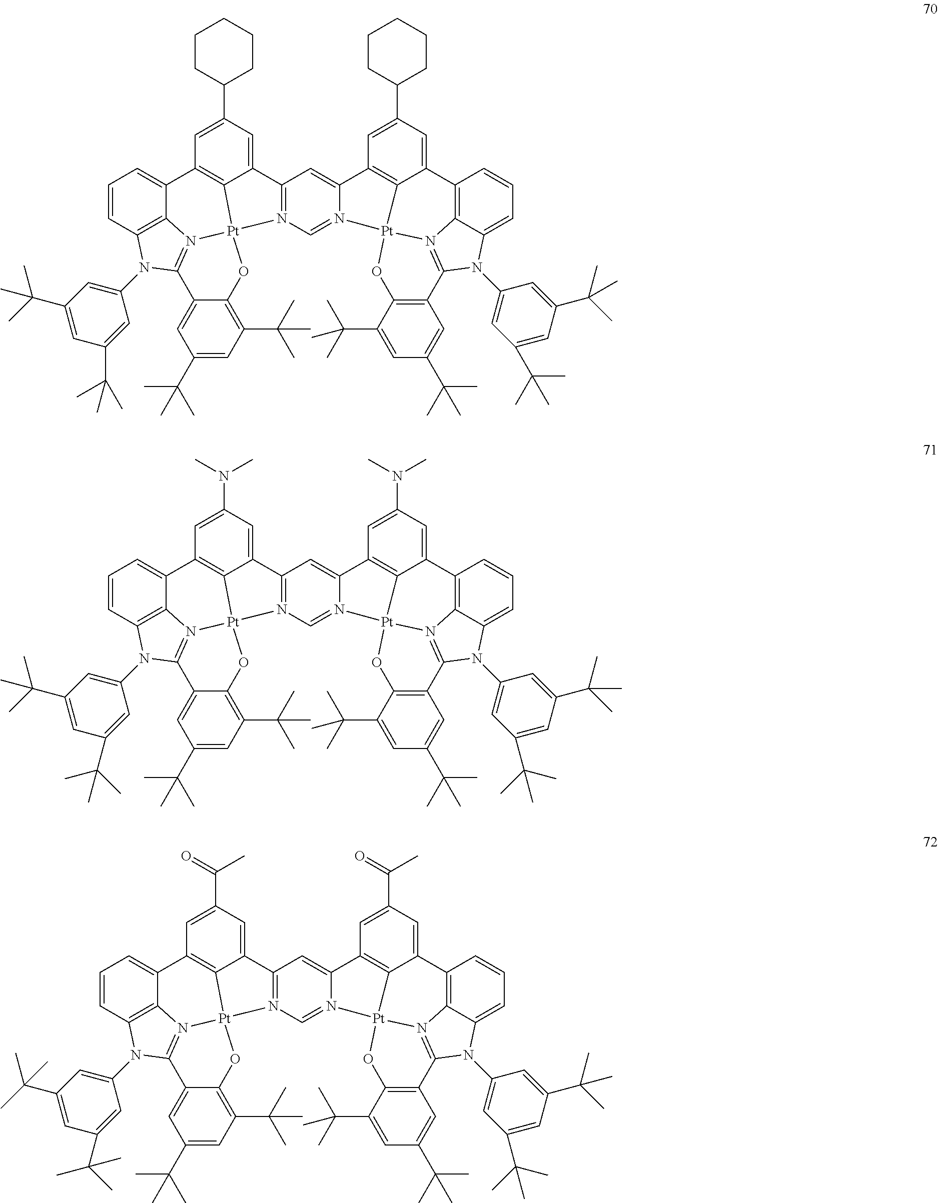

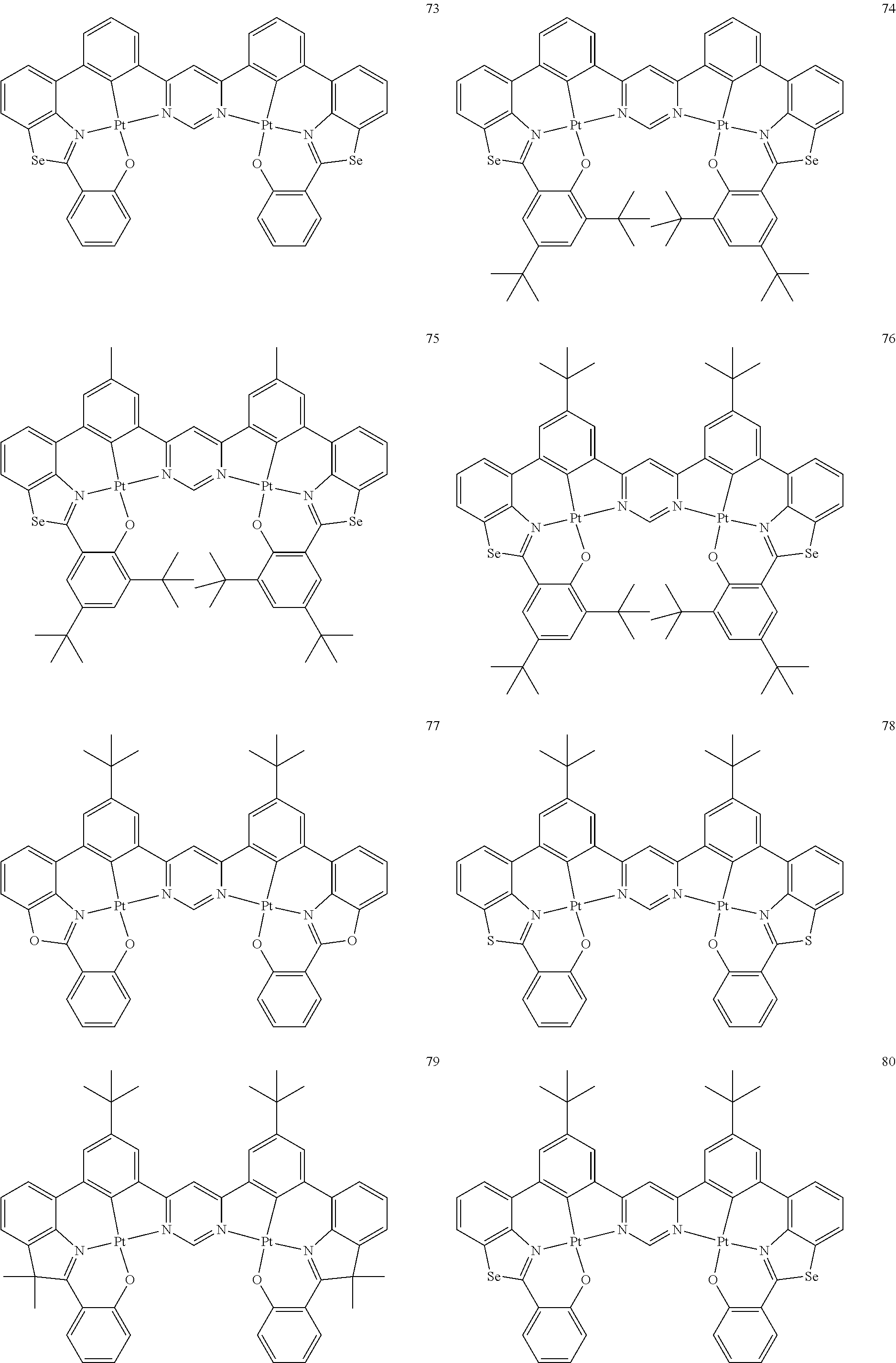

11. A ligand of the binuclear platinum complex according to claim 1, wherein the ligand has the following structural formula: 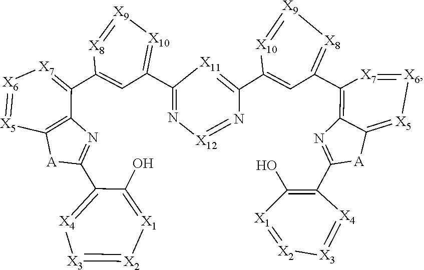
wherein X₁ to X₁₂ and A are defined above.

12. An organic optoelectronic device, comprising: the binuclear platinum complex according to claim 1, wherein the device is an organic light-emitting diode, an organic thin film transistor, an organic photovoltaic device, a light-emitting electrochemical cell, or a chemical sensor.

13. An organic light-emitting diode comprising a cathode, an anode, and an organic layer, wherein
the organic layer is one or more layers selected from a hole injection layer, a hole transport layer, a light-emitting layer, a hole blocking layer, an electron injection layer, or an electron transport layer, and
the organic layer includes the binuclear platinum complex according to claim 1.

14. The organic light-emitting diode according to claim 13, wherein the organic layer including the binuclear platinum complex is the light-emitting layer.
